(19) United States
(12) Patent Application Publication
(10) Pub. No.: US 2025/0065135 A1
(43) Pub. Date: Feb. 27, 2025

(54) ENCLOSURES WITH HERMETIC FEEDTHROUGHS

(71) Applicant: MEDTRONIC, INC., Minneapolis, MN (US)

(72) Inventors: David A. Ruben, Mesa, AZ (US); Caian Qiu, Chandler, AZ (US); Andrew J. Ries, Lino Lakes, MN (US); Andreas Fenner, Chandler, AZ (US)

(21) Appl. No.: 18/725,116

(22) PCT Filed: Dec. 27, 2022
(86) PCT No.: PCT/US2022/054069

§ 371 Date: Jun. 27, 2024

Related U.S. Application Data
(60) Provisional application No. 63/294,189, filed on Dec. 28, 2021.

(51) Int. Cl. A61N 1/375; H01M 50/119; H01M 50/145; H01M 50/186; H01M 50/222; H01M 50/24; H01M 50/247; H01M 50/562

(52) U.S. Cl. CPC A61N 1/3754; H01M 50/119; H01M 50/145; H01M 50/186; H01M 50/222; H01M 50/24; H01M 50/247; H01M 50/562; H01M 2220/30

(57) ABSTRACT

Various embodiments of hermetically-scaled packages and systems are disclosed. The hermetically sealed packages or systems include one or more corrosion-resistant vias disposed in the substrate or housing. Each of the one or more corrosion-resistant vias include one or more sidewalls formed by the substrate or housing, a corrosion-resistant alloy, and a hermetic and corrosion-resistant seal formed between the corrosion-resistant alloy and the one or more sidewalls.

### CROSS-REFERENCE TO RELATED APPLICATION

This application claims the benefit of U.S. Provisional Application No. 63/294,189, filed Dec. 28, 2021, the disclosure of which is incorporated by reference herein in its entirety.

### TECHNICAL FIELD

This disclosure generally relates to hermetically sealed devices and enclosures that house electrical components.

### BACKGROUND

Various systems require electrical coupling between electrical devices disposed within a sealed enclosure or housing and devices or systems external to the enclosure. Oftentimes, such electrical coupling needs to withstand various environmental factors such that a conductive pathway or pathways from the external surface of the enclosure to within the enclosure remains stable. For example, implantable medical devices (IMDs), e.g., cardiac pacemakers, defibrillators, neurostimulators, and drug pumps, which include electronic circuitry and one or more power sources, require an enclosure or housing to contain and seal these elements within a body of a patient. Many of these IMDs include one or more electrical components such as, for example, feedthrough assemblies to provide electrical connections between the elements contained within the housing and components of the IMD external to the housing, for example, one or more sensors, electrodes, and lead wires mounted on an exterior surface of the housing, or electrical contacts housed within a connector header, which is mounted on the housing to provide coupling for one or more implantable leads. Existing electrical components may include a substrate that includes vias filled with copper-based alloys.

### SUMMARY

The techniques of this disclosure generally relate to hermetically sealed and corrosion-resistant packages or enclosures that include corrosion-resistant vias in a substrate. The corrosion-resistant vias are filled with a corrosion-resistant alloy capable of bonding to substrates that include transparent ceramics or sapphire and forming a hermetic seal. Such corrosion-resistant alloys may exhibit increased corrosion resistance and reduced porosity relative to existing conductive via fill materials such as, for example, copper-alloys or other materials susceptible to corrosion. Accordingly, corrosion-resistant vias formed using the methods and materials described herein may allow the construction of packages, devices, and systems that have increased corrosion resistance and long-term hermeticity.

In one example, aspects of this disclosure relate to a hermetically-sealed package. The hermetically-sealed package includes a housing, one or more electronic devices disposed inside the housing, a substrate, and one or more corrosion-resistant vias disposed in the substrate. The substrate includes ceramic, sapphire, or glass. The substrate is hermetically sealed to the housing. Each of the one or more corrosion-resistant vias includes one or more sidewalls formed by the substrate, a corrosion-resistant alloy, and a hermetic and corrosion-resistant seal formed between the corrosion-resistant alloy and the one or more sidewalls.

In another example, aspects of this disclosure relate to a system including a hermetically-sealed battery. The hermetically-sealed battery includes a battery housing, an electrochemical cell, a first electrode, and a second electrode. The battery housing includes ceramic, sapphire, or glass. The electrochemical cell is disposed within the battery housing. The electrochemical cell includes an anode, a cathode, a separator arranged between the anode and the cathode, and an electrolyte. The separator is configured to prevent direct contact between the anode and the cathode. The electrolyte transports positively charged ions between the cathode and the anode. The first electrode includes a corrosion-resistant via disposed in the battery housing and electrically coupled to the anode of the electrochemical cell. The second electrode includes a corrosion-resistant via disposed in the battery housing and electrically coupled to the cathode of the electrochemical cell. The corrosion-resistant via of the first electrode and the corrosion-resistant via of the second electrode each includes one or more sidewalls formed by the battery housing, a corrosion-resistant alloy, and a hermetic and corrosion-resistant seal formed between the corrosion-resistant alloy and the one or more sidewalls formed by the battery housing.

All headings provided herein are for the convenience of the reader and should not be used to limit the meaning of any text that follows the heading, unless so specified.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

In this application, terms such as “a,” “an,” and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.)

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic cross-sectional view of a portion of an apparatus.

FIGS. 2-9 are schematic cross-sectional diagrams depicting various stages of a method or process for forming an electrical component including a substrate, one or more vias in the substrate, and a corrosion-resistant alloy disposed in the one or more vias and bonded to a sidewall of each of the one or more vias.

FIG. 2 is a schematic cross-sectional view of a provided substrate.

FIG. 3 is a schematic cross-sectional view of vias formed in the substrate of FIG. 2.

FIG. 4 is a schematic cross-section view of a corrosion-resistant alloy being disposed on the substrate in the form of a bump array.

FIGS. 5-7 are schematic cross-section views of the corrosion-resistant alloy being disposed on the substrate of FIGS. 2 and 3 using a screen-printed alloy paste that includes the corrosion-resistant alloy.

FIG. 5 is a schematic cross-sectional view of the substrate of FIGS. 2 and 3 with a stencil and alloy paste dispensed prior to being pulled across the stencil.

FIG. 6 is a schematic cross-sectional view of the substrate of FIG. 5 after the alloy paste has been pulled across the stencil.

FIG. 7 is a schematic cross-sectional view of the substrate of FIG. 6 after the stencil has been removed leaving the paste on designated portions of the substrate or in the vias.

FIG. 8 is a schematic cross-sectional view of the substrate of FIGS. 2 and 3 with alloy paste dispensed in the one or more vias and on an outer surface of the substrate proximal to the one or more vias.

FIG. 9 is a schematic cross-sectional view of the electrical component of FIG. 4, 7, or 8 after the corrosion-resistant alloy has been reflowed into the vias.

FIG. 10 is a schematic flow diagram depicting a method or process for forming the electrical component of FIG. 9.

FIG. 11 is a schematic cross-sectional view of a portion of an apparatus.

FIGS. 12-20 are schematic cross-sectional diagrams depicting various stages of a method or process for forming an electrical component including a substrate, one or more vias in the substrate, and a gold alloy disposed in the one or more vias and bonded to a sidewall of each of the one or more vias.

FIG. 12 is a schematic cross-sectional view of a provided substrate.

FIG. 13 is a schematic cross-sectional view of vias formed in the substrate of FIG. 12.

FIG. 14 is a schematic cross-section view of an interface layer disposed on the sidewall of the vias of FIG. 13.

FIG. 15 is a schematic cross-section view of a gold alloy being disposed on the substrate of FIG. 14 in the form of a bump array.

FIGS. 16-18 are schematic cross-section views of the gold alloy being disposed on the substrate of FIG. 14 using a screen-printed alloy paste that includes the gold alloy.

FIG. 16 is a schematic cross-sectional view of the substrate of FIG. 14 with a stencil and alloy paste dispensed prior to being pulled across the stencil.

FIG. 17 is a schematic cross-sectional view of the substrate of FIG. 16 after the alloy paste has been pulled across the stencil.

FIG. 18 is a schematic cross-sectional view of the substrate of FIG. 17 after the stencil has been removed leaving the paste on designated portions of the substrate or in the vias.

FIG. 19 is a schematic cross-sectional view of the substrate of FIGS. 14 with alloy paste dispensed in the one or more vias and on an outer surface of the substrate proximal to the one or more vias.

FIG. 20 is a schematic cross-sectional view of the electrical component of FIG. 15, 18, or 19 after the gold alloy has been reflowed into the vias.

FIG. 21 is a schematic flow diagram depicting a method or process for forming the electrical component of FIG. 20.

FIG. 22 is a schematic cross-sectional view of a hermetically-sealed package that includes corrosion-resistant vias as described herein.

FIG. 23 is a schematic cross-sectional view of a system that includes corrosion-resistant vias as described herein.

### DETAILED DESCRIPTION

In general, the present disclosure provides various packages, enclosures, and systems that include one or more corrosion-resistant vias and methods of forming corrosion-resistant vias. The packages, enclosures, and system and systems described herein may include a hermetically-sealed housing. Such hermetically-sealed housings may be formed from or include substrates. The one or more corrosion-resistant vias may each include one or more sidewalls, a corrosion-resistant alloy, and a corrosion-resistant seal. The one or more sidewalls may be formed by the hermetically-sealed housing or substrate. The corrosion-resistant seal may be formed between the corrosion-resistant alloy and the one or more sidewalls. The methods of disposing and reflowing the corrosion-resistant alloy into vias to form corrosion-resistant vias may reduce porosity and cracking of the via fill materials compared to existing via fill materials and methods. Accordingly, additional sealing steps and materials may not be needed to maintain hermeticity and corrosion-resistance of the vias. Additionally, the lack of additional sealing materials may allow the use of thinner conductive elements on outer surfaces of packages, enclosures, and systems as well as attachment of components directly to the corrosion-resistant vias.

Although existing via fill materials such as, for example, copper-based alloys may exhibit good conductivity, such alloys are generally not corrosion-resistant. Additionally, some existing via fill methods/materials may exhibit a level of porosity that may lead to long-term hermeticity failure. In contrast, the corrosion-resistant vias described herein may form a hermetic seal between the corrosion-resistant alloy fill material and the sidewalls of the vias and may have reduced porosity and cracking of the via fill material.

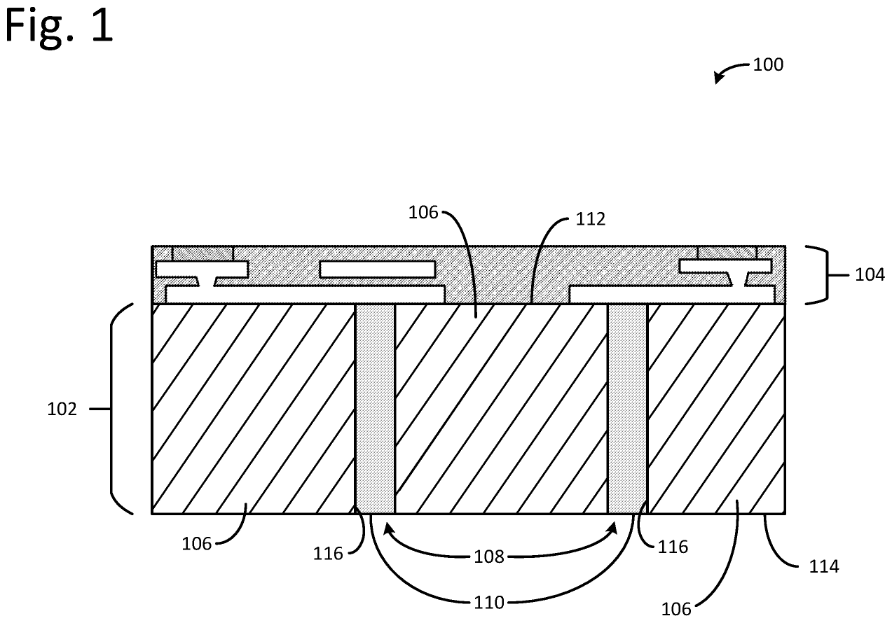

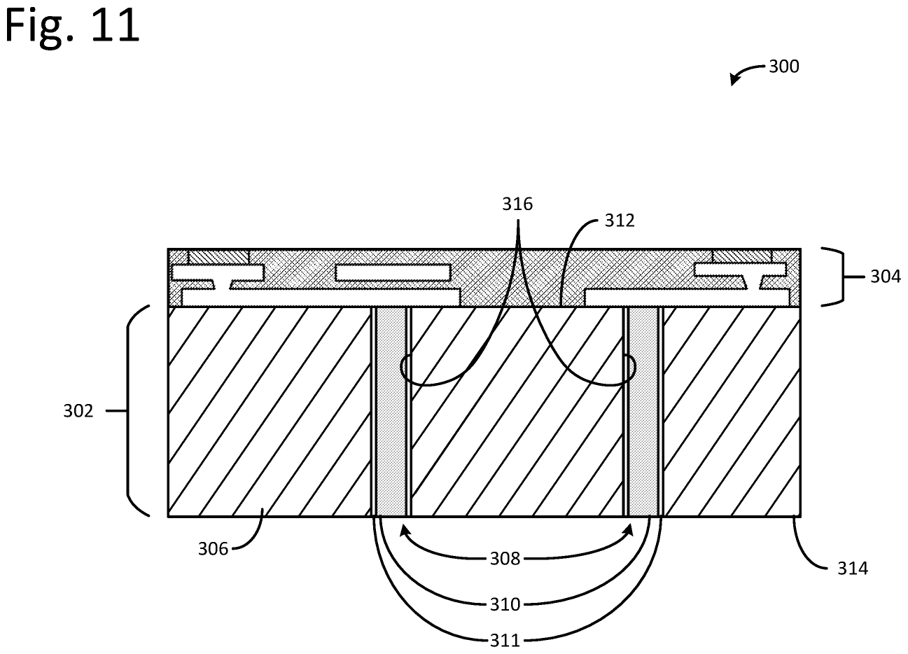

In general, two embodiments of corrosion-resistant vias and methods of forming the same are described herein. The first embodiment is described in regard to FIGS. 1-10. The first embodiment includes corrosion-resistant vias with a corrosion-resistant alloy bonded directly to sidewalls of the vias. The second embodiment is described in regard to FIGS. 11-21. The second embodiment includes corrosion-resistant vias that use an interface layer to bond a corrosion-resistant alloy to sidewalls of the vias.

As used herein, “corrosion resistant” or “corrosion-resistant” may refer to materials, alloys, vias, and layers that exhibit less than a 1 micrometer reduction in height of the outer surface when exposed to a 0.9 percent saline solution at 90 degrees Celsius for 10 weeks. In other words, a layer of material removed from corrosion-resistant materials, alloys, vias, or layers due to exposure to a 0.9 percent saline solution at 90 degrees Celsius for 10 weeks will have a thickness of less than 1 micrometer. In general, corrosion-resistant materials and devices remain chemically stable and resist break down or damage due to chemical processes in corrosive environments such as, e.g., marine environments, underground, in the body (e.g., biostable), etc.

FIG. 1 shows a schematic cross-sectional view of a portion of an apparatus 100 that may be used in sealed packages. The apparatus 100 includes an electrical component 102 and a circuitry 104 disposed on the electrical component 102. The electrical component includes a substrate 106 with vias 108 disposed in the substrate 106. A corrosion-resistant alloy 110 is disposed in the vias 108 and bonded to a sidewall 116 of the vias 108.

The substrate 106 may include any suitable material or materials such as, for example, ceramic, sapphire, or glass. Ceramics may include, for example, alumina (Al₂O₃), nanocrystalline yttria-stabilized zirconia (nc-YSZ), or other biostable ceramics. In at least one embodiment, the substrate 106 includes sapphire. In one or more embodiments, the substrate 106 can include a transparent material. The substrate 106 has a thickness that extends between an outer surface 112 and an outer surface 114. The outer surface 112 may be referred to interchangeably as a first major surface and the outer surface 114 may be referred to interchangeably as a second major surface. The substrate 106 may take on any suitable shape or shapes and have any suitable dimensions.

The vias 108 extend from the outer surface 112 to the outer surface 114 and are exposed at such surfaces. Accordingly, the electrical component 102 may be used as a feedthrough, an interposer, or other electrical component. The vias 108 may have any suitable cross-sectional shape or shapes. For example, the vias 108 may have an elliptical cross section. Such elliptical vias may have a single sidewall 116 that defines the outer diameter of the elliptical vias. Further, for example, the vias 108 may have a polygonal cross section. Such polygonal vias may have three or more sidewalls 116. Still further, for example, the vias 108 may have a cross-sectional shape that includes both straight and curved edges such as, for example, a semicircle, a quadrant, arcs, or combinations of curved and polygonal shapes. Vias with such cross-sectional shapes may include two or more sidewalls 116.

The vias 108 are filled with a corrosion-resistant alloy 110. The corrosion-resistant alloy 110 may be bonded to the sidewalls 116. Such bond between the corrosion-resistant alloy 110 and the sidewalls 116 may provide a hermetic seal. The corrosion-resistant alloy 110 may include any suitable material or materials. Such materials may include one or more of, for example, titanium, niobium, nickel, hafnium, etc. In at least one embodiment, the corrosion-resistant alloy 110 includes zirconium alloys. Zirconium alloys may include, for example, Z-61Zr, Z-62Zr, or Z-64Zr. The zirconium alloys may refer to AUFHAUSER ZIRCONIUMALLOYS Z-61Zr or Z-62Zr. The zirconium alloy Z-61Zr may include to 58 to 64 percent by weight zirconium (Zr), 19 to 21 percent by weight nickel (Ni), 10 to 11 percent by weight titanium (Ti), 6 to 8 percent by weight niobium (Nb), and 1 to 2 percent hafnium (Hf). The zirconium alloy Z-62Zr may include to 59.5 to 64.9 percent by weight zirconium (Zr), 19 to 21 percent by weight nickel (Ni), 16 to 18 percent by weight titanium (Ti), and 0.1 to 1.5 percent hafnium (Hf). In at least one other embodiment, the corrosion-resistant alloy 110 includes a titanium alloy TiNi67.

As used herein, “corrosion-resistant” or “corrosion-resistant” may refer to materials, alloys, vias, and layers that exhibit less than a 1 micrometer reduction in height of the outer surface when exposed to a 0.9 percent saline solution at 90 degrees Celsius for 10 weeks. In other words, a layer of material removed from corrosion-resistant materials, alloys, vias, or layers due to exposure to a 0.9 percent saline solution at 90 degrees Celsius for 10 weeks will have a thickness of less than 1 micrometer. In general, corrosion-resistant materials and devices remain chemically stable and resist break down or damage due to chemical processes in corrosive environments such as, e.g., marine environments, underground, in the body (e.g., biostable), etc. Accordingly, vias such as vias 108 that are filled with the corrosion-resistant alloy 110 may not include alloys or compositions that may compromise a corrosion-resistance or a biostability of the vias. In other words, vias filled with the corrosion-resistant alloy 110 may be corrosion-resistant vias. While not all alloys that include titanium, niobium, nickel, hafnium, and other elements may be corrosion-resistant, the corrosion-resistant alloys described herein refer to a subset of such alloys that are corrosion-resistant.

The circuitry 104 may include any suitable circuitry or components for incorporating the electrical component 102 in a device. The circuitry 104 may include, for example, multiple layers, substrates, conductive traces, vias, passive components, active components, pads, electrodes, or other electrical components. The circuitry 104 may be soldered or otherwise sealed to the substrate 106. The circuitry 104 may take on any suitable shape or shapes and have any suitable dimensions. Generally, the circuitry 104 may be shaped to fit in a housing of a device.

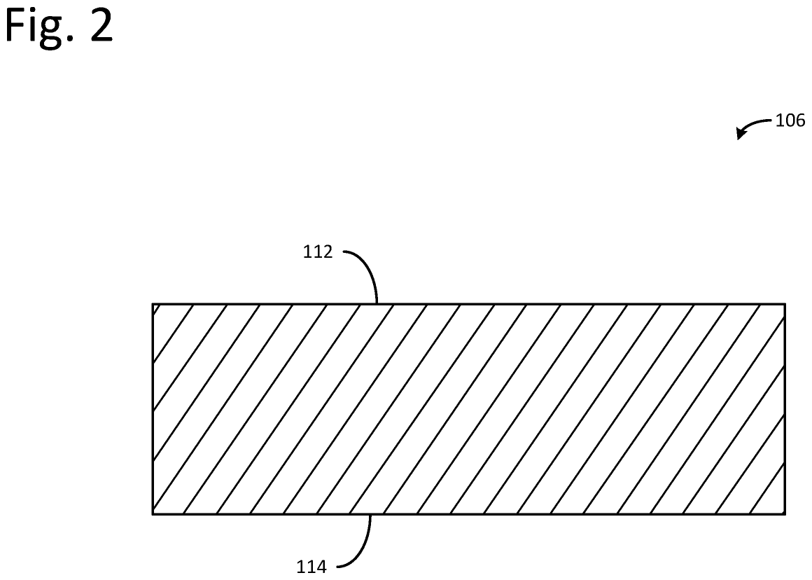

FIGS. 2-9 show various stages of various methods or processes of forming an electrical component such as, for example, electrical component 102. Although such methods and processes are described in reference to electrical component 102 of FIG. 1, the methods and processes may be used for any suitable electrical component.

FIG. 2 shows the substrate 106 provided prior to vias 108 being formed in the substrate. The substrate 106 extends between the outer surface 112 and the outer surface 114. The substrate may include ceramic or sapphire. Additionally, providing the substrate 106 may include any suitable preparatory steps such as, for example, shaping, grinding, or polishing the outer surfaces 112, 114 of the substrate 106.

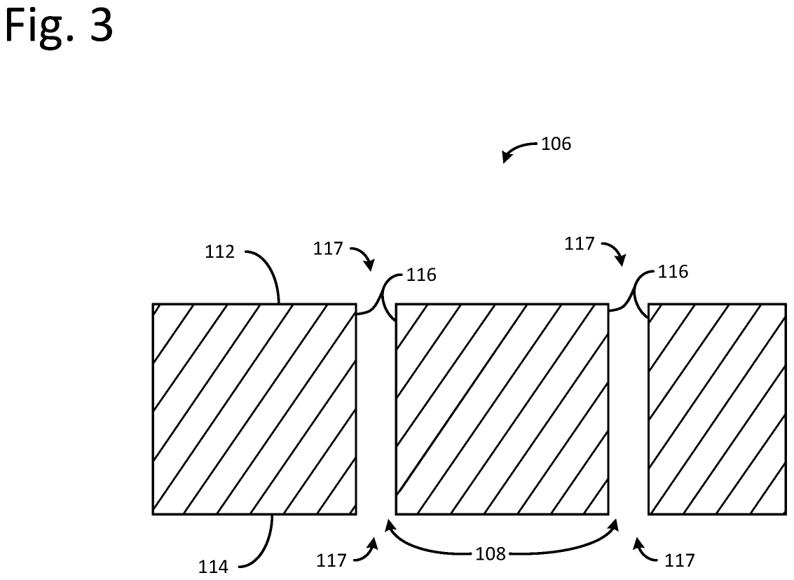

FIG. 3 shows the substrate 106 after the vias 108 have been formed. Each of the vias 108 include openings 117 and extend from an opening 117 at the outer surface 112 to an opening 117 at the outer surface 114. Additionally, the vias 108 include a sidewall 116 formed by the substrate 106. The vias 108 may be formed using any suitable technique or techniques. For example, the vias 108 may be formed by laser, mechanical drilling, or etching.

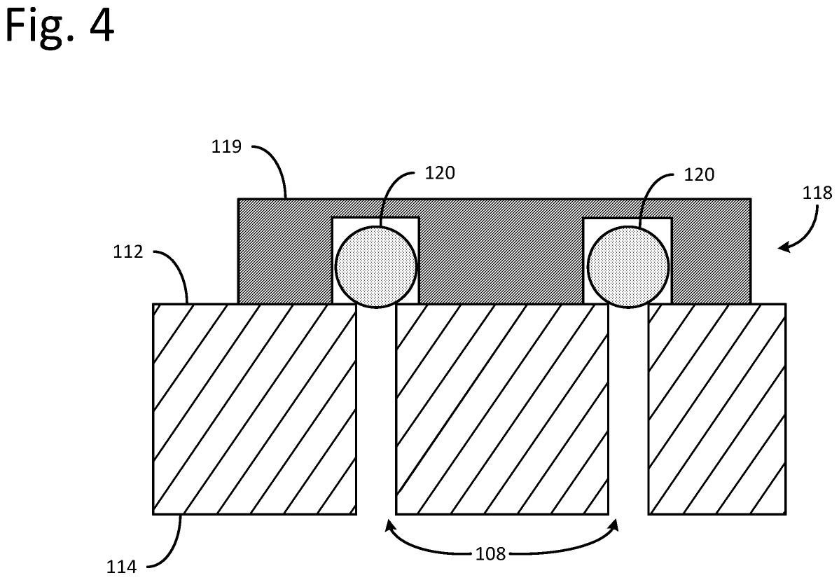

FIGS. 4 shows a bump array 118 being used to dispose the corrosion-resistant alloy 110 on the substrate 106. The bump array 118 includes a stencil or base 119 and alloy bumps 120. The stencil 119 may be used to secure the position of the alloy bumps 120 relative to the vias 108 prior to a reflowing process. The stencil 119 may include any suitable materials such as, for example, graphite, silicon, stainless steel, anodized aluminum, etc. In at least one embodiment, the stencil 119 includes a graphite sheet. The stencil 119 may be removed prior to or after reflow of the corrosion-resistant alloy 110.

The alloy bumps 120 include the corrosion-resistant alloy 110. The alloy bumps 120 may further include any suitable material or materials to aid a reflow process. For example, the alloy bumps 120 may include flux, organic binders, fluid, etc. Such materials may aid the corrosion-resistant alloy 110 in filling the vias 108 and bonding to the sidewalls 116. Furthermore, binders and/or fluid may facilitate maintaining the position of the alloy bumps 120 prior to reflow. The alloy bumps 120 may take one any suitable shape or shapes. For example, the alloy bumps 120 may be substantially spherical, discoid, parallelepiped, hemispherical, or other suitable shape. Each of the alloy bumps 120 may be of a size sufficient to fill the vias 108 during a reflow process.

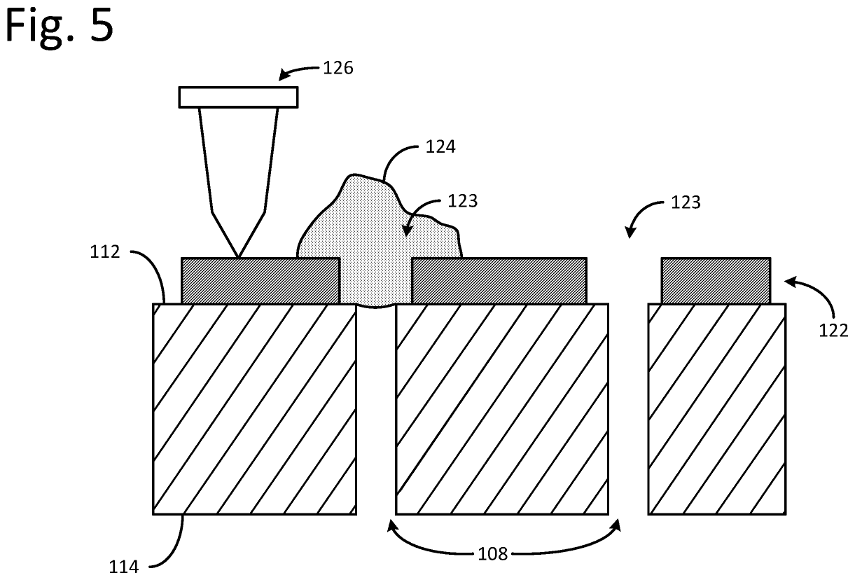

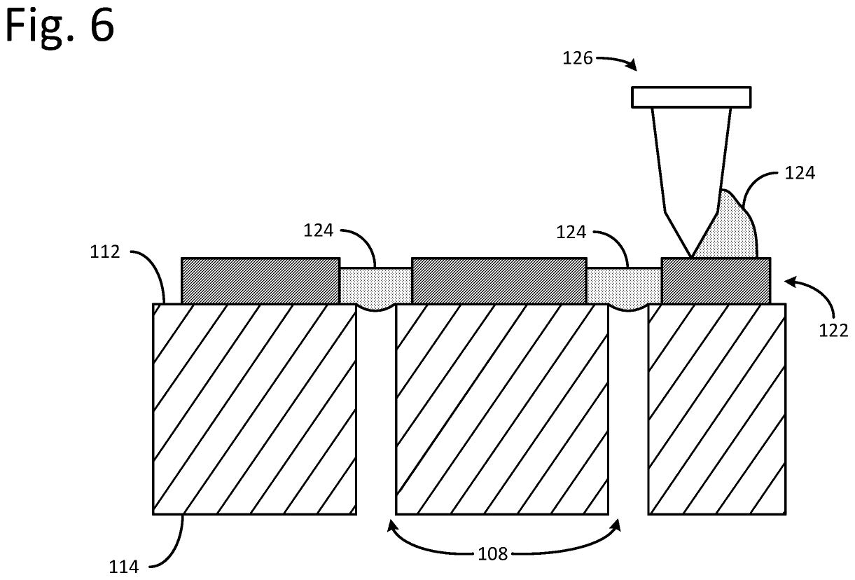

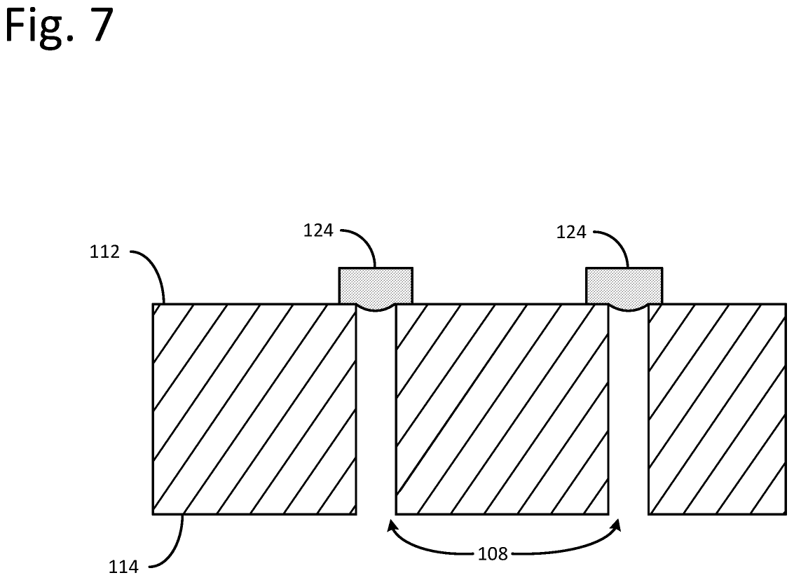

FIGS. 5-7 show a stencil-printing process being used to dispose the corrosion-resistant alloy 110 on the substrate 106. The stencil-printing process may include positioning a stencil 122 on the outer surface 112 of the substrate 106, disposing alloy paste 124 at one side of the stencil 122, pulling the alloy paste 124 across the stencil 122 and the substrate 106, and removing the stencil 122 from the substrate 106. FIG. 5 shows the stencil-printing process after the alloy paste 124 has been disposed but before the paste has been pulled across the stencil 122 and the substrate 106. FIG. 6 shows the stencil-printing process after the alloy paste 124 has been pulled across the stencil 122 and the substrate 106 but before the stencil has been removed from the substrate 106. FIG. 7 shows the substrate 106 and the alloy paste 124 after the stencil has been removed and the stencil-printing process is complete.

The stencil 122 may be used to secure the position of the alloy paste 124 relative to the vias 108 prior to a reflowing step or process. For example, the stencil 122 may include openings 123 at or near the position of the vias 108. The openings 123 may allow the alloy paste 124 to be deposited in the vias 108 or on the outer surface 112 of the substrate 106 proximal to the vias 108. The stencil 122 may include any suitable materials such as, for example, stainless steel, nickel, etc. In at least one embodiment, the stencil 119 includes stainless steel.

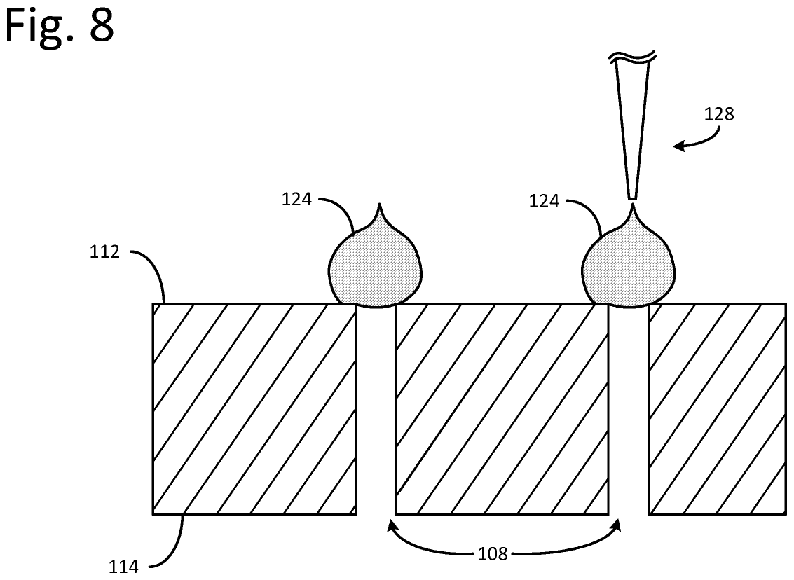

The alloy paste 124 may include the corrosion-resistant alloy 110. The corrosion-resistant alloy 110 may be included in the alloy paste 124 as alloy particles. The alloy paste 124 can include binding agents to hold the alloy particles together. The alloy paste 124 can include any suitable binding agents, e.g., organic binders, solvents, etc. The alloy paste 124 can be dispensed using any suitable dispensing tools and/or nozzles such as, for example, dispenser 128 (see FIG. 8).

The alloy paste 124 can be pulled across the stencil 122 and the substrate 106 using any suitable tool or tools. For example, the alloy paste 124 can be pulled across the stencil 122 and the substrate 106 using a squeegee 126. The squeegee 126 may be configured to pull the alloy paste 124 across the stencil 122 and the substrate 106 causing the alloy paste 124 to be deposited in the openings 123 of the stencil 122. After the alloy paste 124 has been deposited in the openings 123 of the stencil 122, the stencil 122 can be removed from the outer surface 112 of the substrate 106 leaving the alloy paste 124 positioned in and proximal to the vias 108. Accordingly, the substrate 106 and the alloy paste 124 of FIG. 7 may be ready for a reflow process for reflowing the corrosion-resistant alloy 110 into the vias 108.

FIG. 8 shows a dispensing process being used to dispose the corrosion-resistant alloy 110 on the substrate 106. As shown, the dispenser 128 is used to dispense alloy paste 124 onto the substrate 106 proximal to, over, and in the vias 108. The dispenser 128 may be configured to dispense an amount of the alloy paste 124 sufficient to fill the vias 108 with the corrosion-resistant alloy 110 during a reflow process. Accordingly, the substrate 106 and the alloy paste 124 of FIG. 8 may be ready for a reflow process for reflowing the corrosion-resistant alloy 110 into the vias 108.

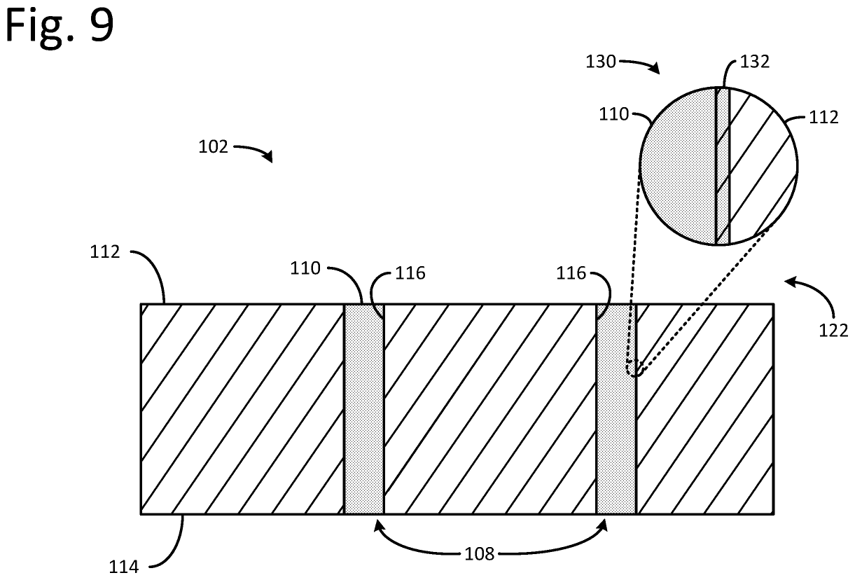

FIG. 9 shows the electrical component 102 after reflowing the corrosion-resistant alloy 110 into the vias 108. The corrosion-resistant alloy 110 filling the vias 108 as depicted in FIG. 9 can be produced by reflowing the alloy bumps 120 of FIG. 4 or the alloy paste 124 of FIGS. 5-8. The electrical component 102 includes a reaction layer 132 that may be adapted to bond the corrosion-resistant alloy and the sidewall 116 of each of the one or more vias 108. The reaction layer 132 may be formed between the sidewalls 116 of the vias 108 and the corrosion-resistant alloy 110 as shown in the zoomed in portion 130. Such reaction layer 132 may be formed during a reflow process. The reaction layer 132 may be formed when the corrosion-resistant alloy 110 bonds with oxygen of the substrate 106 that form the sidewalls 116 of the vias 108. Such bond or reaction layer 132 may provide a hermetic seal.

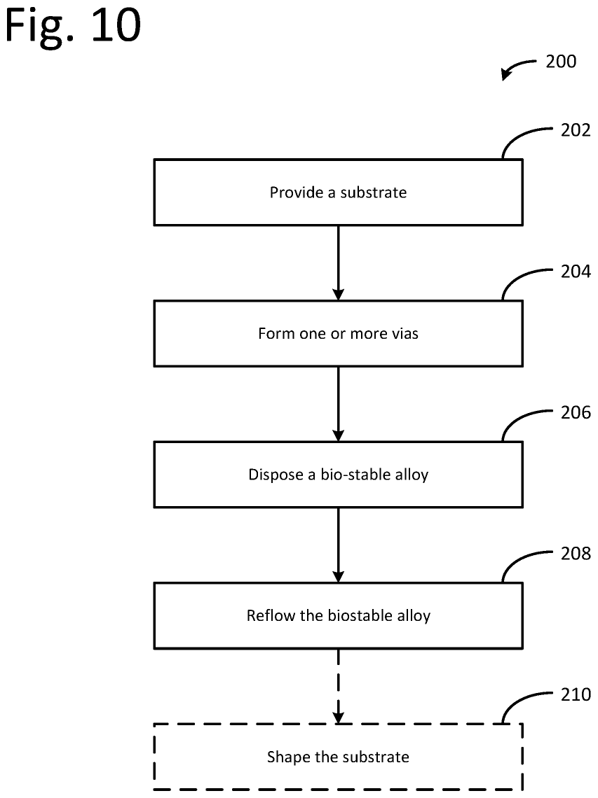

FIG. 10 shows a schematic flow diagram of a method or process 200 for forming an electrical component such as, for example, electrical component 102. Although described in reference to electrical component 102 of FIGS. 1 and 9, the process 200 may be used for any suitable electrical component that includes a substrate, one or more vias, and a corrosion-resistant alloy disposed in the vias.

At 202, the substrate 106 may be provided (see FIG. 2). The substrate may include transparent ceramic or sapphire. Providing the substrate 106 may include shaping, grinding, or polishing to form the outer surface 112 and the outer surface 114.

At 204, one or more vias 108 may be formed in the substrate 106 (see FIG. 3). Each of the one or more vias 108 may include an opening 117 at an outer surface 112 of the substrate 106. Additionally, each of the one or more vias 108 may include a sidewall 116 formed by the substrate 106. The one or more vias 108 may be formed using any suitable technique or techniques. For example, the one or more vias may be formed using laser or mechanical drilling.

At 206, the corrosion-resistant alloy 110 may be disposed in the one or more vias 108 or on the outer surface 112 of the substrate 106 proximal to the one or more vias 108. As used herein, the term “proximal to the one or more vias” may refer to being within 30 mm of the openings 117 of the one or more vias 108. Additionally, the corrosion-resistant alloy may be disposed on the outer surface 112 of the substrate such that the opening 117 of each of the one or more vias 108 is at least partially covered by the corrosion-resistant alloy 110.

The corrosion-resistant alloy 110 may be disposed using any suitable technique or techniques. For example, disposing the corrosion-resistant alloy may include, but is not limited to, disposing a bump array 118 of the corrosion-resistant alloy 110 on the outer surface 112 of the substrate 106 such that the opening 117 of each of the one or more vias 108 is at least partially covered by an alloy bump 120 of the bump array 118 (see FIG. 4). The alloy bumps 120 can be held in place using any suitable technique. In one or more embodiments, the alloy bumps 120 of the bump array 118 may be held in place by a stencil 119 while the bump array 118 is disposed on the outer surface 112 of the substrate 106. In one or more embodiments, the alloy bumps 120 can be held in place, e.g., with flux, paste, fluid, etc. Further, for example, disposing the corrosion-resistant alloy 110 may include screen printing alloy paste 124 including the corrosion-resistant alloy 110 in the one or more vias 108 or on the outer surface 112 of the substrate 106 proximal to the one or more vias 108 (see FIGS. 5-7). Still further, for example, disposing the corrosion-resistant alloy includes dispensing an alloy paste including the corrosion-resistant alloy in the one or more vias or on the outer surface of the substrate proximal to the one or more vias (see FIG. 8). Additionally, other techniques for disposing via fill material may be used to dispose the corrosion-resistant alloy 110 in the one or more vias 108 or on the outer surface 112 of the substrate 106 proximal to the one or more vias 108.

At 208, the corrosion-resistant alloy 110 may be reflowed into the one or more vias 108. Reflowing the corrosion-resistant alloy 110 may include reducing an atmospheric pressure around the substrate 106 and the corrosion-resistant alloy 110. The atmospheric pressure may be reduced to any suitable level, for example, from at least 10⁻⁷ Torr to no greater than 10⁻⁵ Torr. In at least one embodiment, the atmospheric pressure is reduced to less than 10⁻⁶ Torr.

Reflowing the corrosion-resistant alloy 110 may include brazing the substrate 106 and the corrosion-resistant alloy 110. Reflowing the corrosion-resistant alloy 110 may include heating the substrate 106 and the corrosion-resistant alloy 110 to a peak temperature above the liquidus temperature of the corrosion-resistant alloy 110. The liquidus temperature of the corrosion-resistant alloy 110 may be the temperature at which the corrosion-resistant alloy forms solids within a few hours and remains in equilibrium with liquids. The exact liquidus temperature of the corrosion-resistant alloy 110 may depend on its composition (e.g., Z-61Zr, Z-62Zr, TiNi67, or other corrosion-resistant alloy composition). Reflowing the corrosion-resistant alloy 110 may include heating the substrate 106 and the corrosion-resistant alloy 110 to a peak temperature, for example, of at least 10 degrees Celsius greater than the liquidus temperature of the corrosion-resistant alloy 110 and no greater than 150 degrees Celsius greater than the liquidus temperature of the corrosion-resistant alloy 110 or to a peak temperature within any suitable range therebetween. For example, reflowing the corrosion-resistant alloy 110 may include heating the substrate 106 and the corrosion-resistant alloy 110 to a peak temperature in a range from at least 10 degrees Celsius, 15 degrees Celsius, 20 degrees Celsius, 25 degrees Celsius, 30 degrees Celsius, 35 degrees Celsius, 40 degrees Celsius, 45 degrees Celsius, or 50 degrees Celsius greater than the liquidus temperature to no greater than 100 degrees Celsius, 105 degrees Celsius, 110 degrees Celsius, 115 degrees Celsius, 120 degrees Celsius, 125 degrees Celsius, 130 degrees Celsius, 135 degrees Celsius, 140 degrees Celsius, 145 degrees Celsius, or 150 degrees Celsius greater than the liquidus temperature. In at least one embodiment, reflowing the corrosion-resistant alloy 110 may include heating the substrate 106 and the corrosion-resistant alloy 110 to a peak temperature of at least 50 degrees Celsius greater than the liquidus temperature of the corrosion-resistant alloy 110 and no greater than 100 degrees Celsius greater than the liquidus temperature of the corrosion-resistant alloy 110.

Reflowing the corrosion-resistant alloy 110 may include heating the substrate 106 and the corrosion-resistant alloy 110 to a peak temperature that is independent from the liquidus temperature. In at least one embodiment, reflowing the corrosion-resistant alloy 110 may include heating the substrate 106 and the corrosion-resistant alloy 110 to a peak temperature of at least 950 degrees Celsius and no greater than 1050 degrees Celsius. Reflowing the corrosion-resistant alloy 110 may include heating the substrate 106 and the corrosion-resistant alloy 110 at the peak temperature, for example, for at least 10 seconds and no greater than 30 minutes or for any suitable range of time therebetween. For example, reflowing the corrosion-resistant alloy 110 may include heating the substrate 106 and the corrosion-resistant alloy 110 at the peak temperature for a time period in a range from at least 10 seconds, 20 seconds, 30 seconds, 40 seconds, 50 seconds, 1 minute to no greater than 3 minutes, 5 minutes, 10 minutes, 15 minutes, 20 minutes, 25 minutes, or 30 minutes. In at least one embodiment, reflowing the corrosion-resistant alloy 110 may include heating the substrate 106 and the corrosion-resistant alloy 110 at the peak temperature for at least 1 minute and no greater than 15 minutes.

Reflowing the corrosion-resistant alloy 110 may form a reaction layer 132 between the corrosion-resistant alloy 110 and the substrate 106. Heating the corrosion-resistant alloy 110 to suitable melting temperatures may allow the corrosion-resistant alloy 110 to displace atoms (e.g., aluminum atoms of substrates that include Al₂O₃) in the substrate 106 and bond to oxygen atoms of the substrate 106. Such displacement and bonding may form the reaction layer 132.

At 210, the substrate 106 may be shaped. Shaping the substrate 106 may include grinding or polishing one or more surfaces of the substrate 106. Additionally, shaping of the substrate 106 may include grinding or polishing the one or more vias 108. Shaping the substrate 106 may smooth portions of the outer surfaces 112, 114 of the substrate 106 and/or the one or more vias 108. Shaping the substrate 106 may result in one or more planar or curved surfaces.

FIG. 11 shows a schematic cross-sectional view of a portion of an apparatus 300 that may be used in sealed packages. The apparatus 300 includes an electrical component 302 and a circuitry 304 disposed on the electrical component 302. The electrical component includes a substrate 306 with vias 308 disposed in the substrate 306. An interface layer 311 is disposed on sidewalls 316 of the vias 308. A gold alloy 310 is disposed in the vias 308 and may be bonded to the sidewalls 316 of the vias 308.

The substrate 306 may include any suitable material or materials such as, for example, ceramic, sapphire, glass, or a semiconductor. Ceramics may include, for example, alumina (Al₂O₃), nanocrystalline yttria-stabilized zirconia (nc-YSZ), or other corrosion-resistant ceramics. In at least one embodiment, the substrate 306 includes sapphire. The substrate 306 has a thickness that extends between an outer surface 312 and an outer surface 314. The outer surface 312 may be referred to interchangeably as a first major surface and the outer surface 314 may be referred to interchangeably as a second major surface. The substrate 306 may take on any suitable shape or shapes and have any suitable dimensions.

The vias 308 extend from the outer surface 312 to the outer surface 314 and are exposed at such surfaces. Accordingly, the electrical component 302 may be used as a feedthrough, an interposer, or other electrical component. The vias 308 may have any suitable cross-sectional shape or shapes. For example, the vias 308 may have an elliptical cross section. Such elliptical vias may have a single sidewall 316 that defines the outer diameter of the elliptical vias. Further, for example, the vias 308 may have a polygonal cross section. Such polygonal vias may have three or more sidewalls 316. Still further, for example, the vias 308 may have a cross-sectional shape that includes both straight and curved edges such as, for example, a semicircle, a quadrant, arcs, or combinations of curved and polygonal shapes. Vias with such cross-sectional shapes may include two or more sidewalls 316.

The interface layer 311 is disposed on the sidewalls 316 of the vias 308. The interface layer 311 may be bonded to the sidewalls 316 formed by the substrate 306. The interface layer 311 may include any suitable material or materials for bonding to the sidewalls 316 of the substrate 306 and the gold alloy 310. Such materials may include, titanium, niobium, gold, platinum, tantalum, zirconium, etc. The interface layer 311 may be corrosion resistant. Accordingly, the interface layer 311 may not include alloys or compositions that may compromise a corrosion resistance of the interface layer 311. While not all alloys that include materials such as titanium, titanium, niobium, gold, platinum, tantalum, and zirconium are corrosion-resistant, corrosion-resistant alloys described herein, may refer to the subset of such materials and alloys that are corrosion resistant.

The vias 308 are filled with the gold alloy 310. The gold alloy 310 may be bonded to the sidewalls 316. The vias 308 may be hermetically sealed by the interface layer 311 and the gold alloy 310. In other words, the bonds between the gold alloy 310, the interface layer 311, and the sidewalls 316 may provide a hermetic seal. The gold alloy 310 may include any suitable material or materials. Such materials may include one or more of, for example, titanium, tin, germanium, niobium, silicon, silver, tungsten, etc. In at least one embodiment, the gold alloy 310 includes Au-20Sn. In at least one other embodiment, the gold alloy 310 includes AuSi. In at least one other embodiment, the gold alloy 310 includes titanium. The gold alloy 310 may be corrosion resistant. Accordingly, the gold alloy 310 may not include alloys or compositions that may compromise a biostability of the gold alloy 310. While not all alloys that include gold and other elements are corrosion resistant, the gold alloys described herein refer to the subset of such alloys that are corrosion resistant. Accordingly, vias, such as vias 308, that are filled with the gold alloy 310 and the interface layer 311 may be corrosion-resistant vias.

The circuitry 304 may include any suitable circuitry or components for incorporating the electrical component 302 in a device (e.g., an implantable medical device). The circuitry 304 may include, for example, multiple layers, substrates, conductive traces, vias, passive components, active components, pads, electrodes, or other electrical components. The circuitry 304 may be soldered or otherwise sealed to the substrate 306. The circuitry 304 may take on any suitable shape or shapes and have any suitable dimensions. Generally, the circuitry 304 may be shaped to fit in a housing of a device.

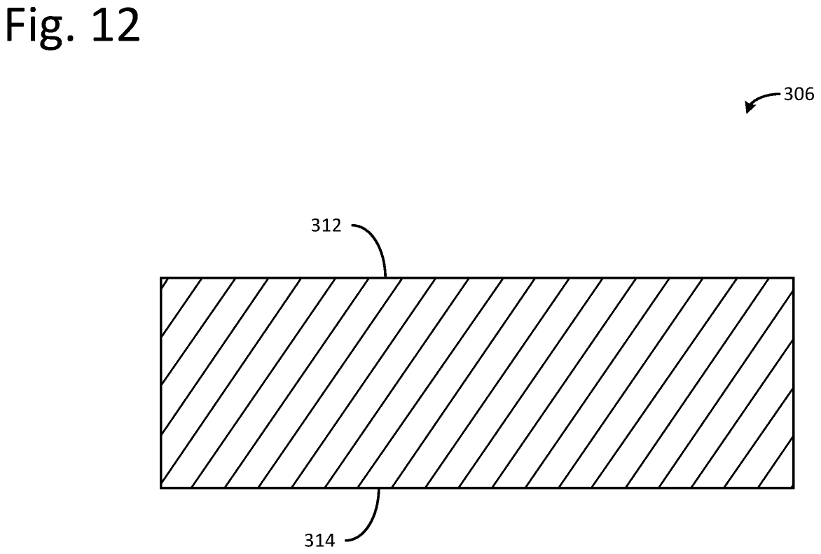

FIGS. 12-20 show various stages of various methods or processes of forming an electrical component such as, for example, electrical component 302. Although such methods and processes are described in reference to electrical component 302 of FIG. 11, the methods and processes may be used for any suitable electrical component that includes a substrate, one or more vias, and a gold alloy disposed in the vias.

FIG. 12 shows the substrate 306 provided prior to vias 308 being formed in the substrate. The substrate 306 extends between the outer surface 312 and the outer surface 314. The substrate may include ceramic or sapphire. Additionally, providing the substrate 306 may include any suitable preparatory steps such as, for example, shaping, grinding, or polishing the outer surfaces 312, 314 of the substrate 306.

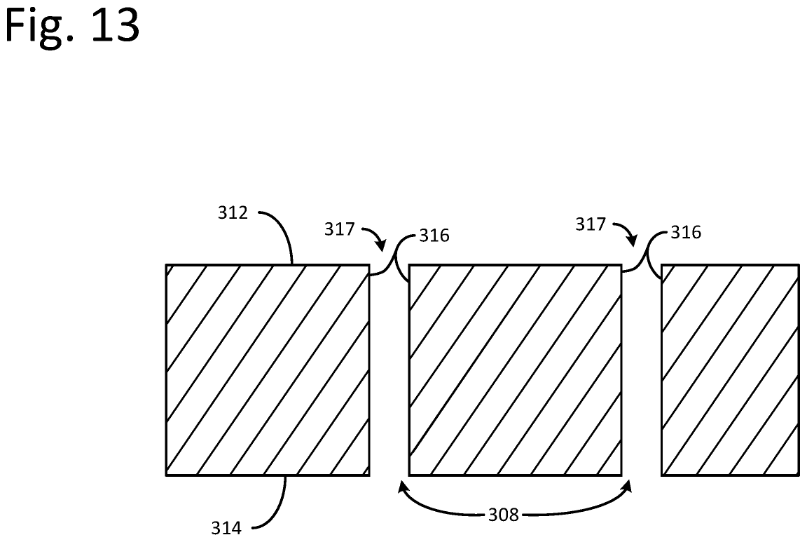

FIG. 13 shows the substrate 306 after the vias 308 have been formed. Each of the vias 308 include openings 317 and extend from an opening 317 at the outer surface 312 to an opening 317 at the outer surface 314. Additionally, the vias 308 include a sidewall 316 formed by the substrate 306. The vias 308 may be formed using any suitable technique or techniques. For example, the vias 308 may be formed by laser or mechanical drilling, or etching.

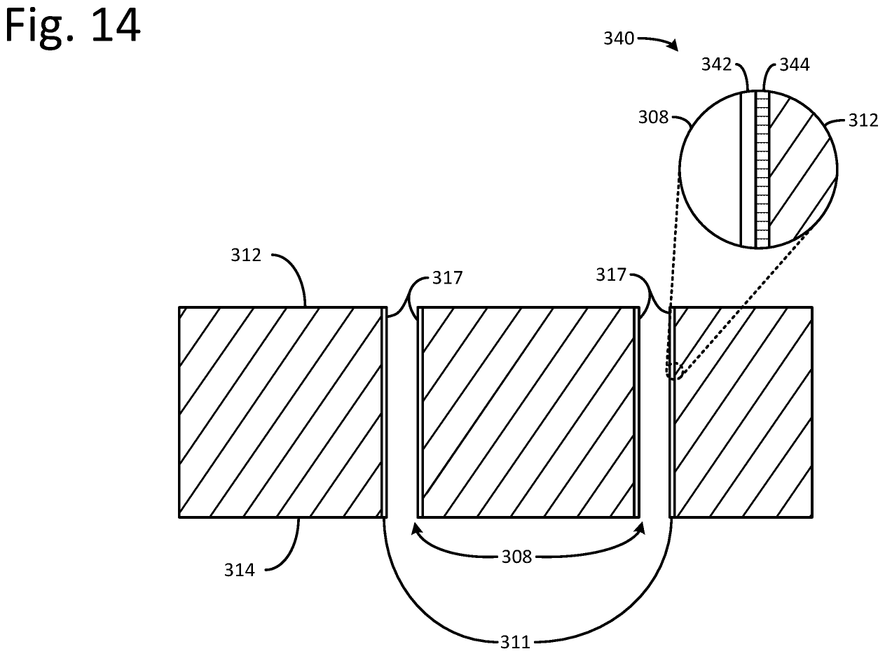

FIG. 14 shows the interface layer 311 disposed on the sidewall of the vias 308 of FIG. 13. The interface layer 311 may include a wettable layer 342 and an adhesion layer 344 as shown in the zoomed in portion 340. The adhesion layer 344 may be disposed on the sidewalls 316 of the vias 308 and the wettable layer 342 may be disposed on the adhesion layer 344. The adhesion layer 344 may include any suitable material or materials for bonding to the sidewalls 316 formed by the substrate 306 during a reflow process. For example, the adhesion layer 344 may include titanium, niobium, platinum, zirconium, tantalum, tungsten, or alloys thereof. The wettable layer 342 may include any suitable material or materials for bonding the gold alloy 310 to the interface layer 311 during a reflow process. For example, the wettable layer 342 may include gold, copper, platinum, palladium, silver, nickel, or alloys thereof. Additionally, the wettable layer 342 and the adhesion layer 344 may be configured to bond to each other during deposition.

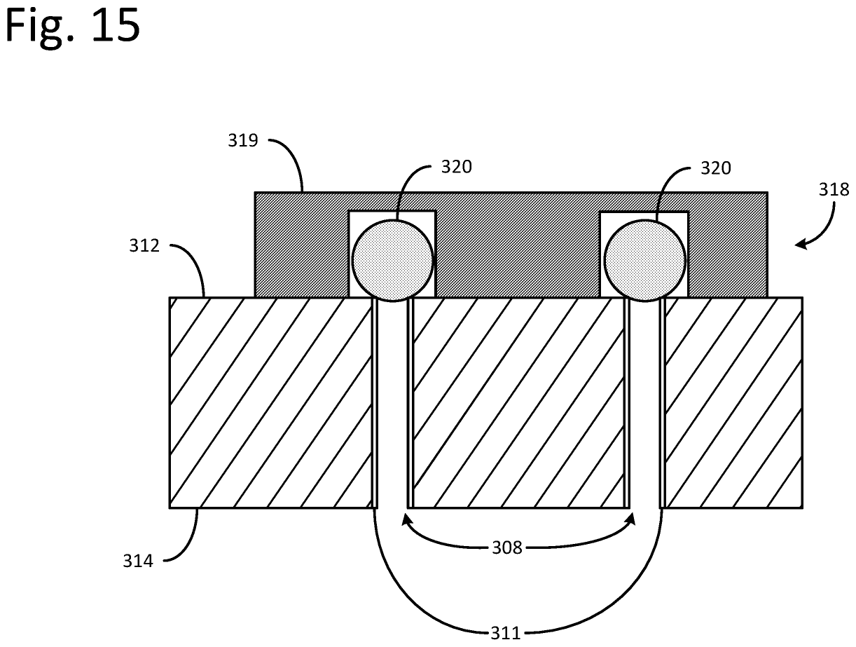

FIG. 15 shows a bump array 318 being used to dispose the gold alloy 310 on the substrate 306. The bump array 318 includes a stencil or base 319 and alloy bumps 320. The stencil 319 may be used to secure the position of the alloy bumps 320 relative to the vias 308 prior to a reflowing process. The stencil 319 may include any suitable materials such as, for example, graphite, silicon, stainless steel, anodized aluminum, etc. In at least one embodiment, the stencil 319 includes a graphite sheet.

The alloy bumps 320 include the gold alloy 310. The alloy bumps 320 may further include any suitable material or materials to aid a reflow process. For example, the alloy bumps 320 may include flux, organic binders, fluid, etc. Such materials may aid the gold alloy 310 in filling the vias 308 and bonding to the sidewalls 316. The alloy bumps 320 may take one any suitable shape or shapes. For example, the alloy bumps 320 may be substantially spherical, discoid, parallelepiped, hemispherical, or other suitable shape. Each of the alloy bumps 320 may be of a size sufficient to fill the vias 308 during a reflow process.

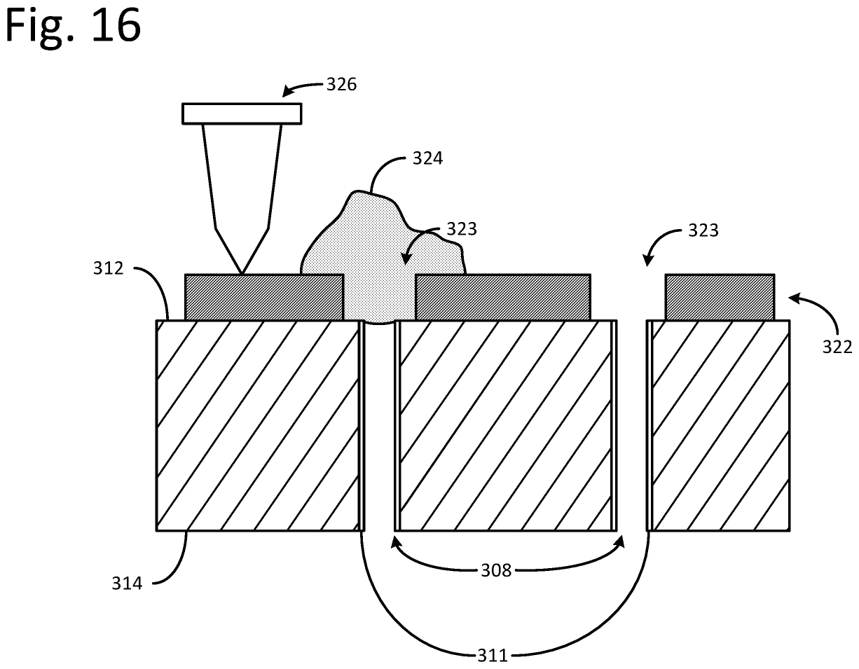

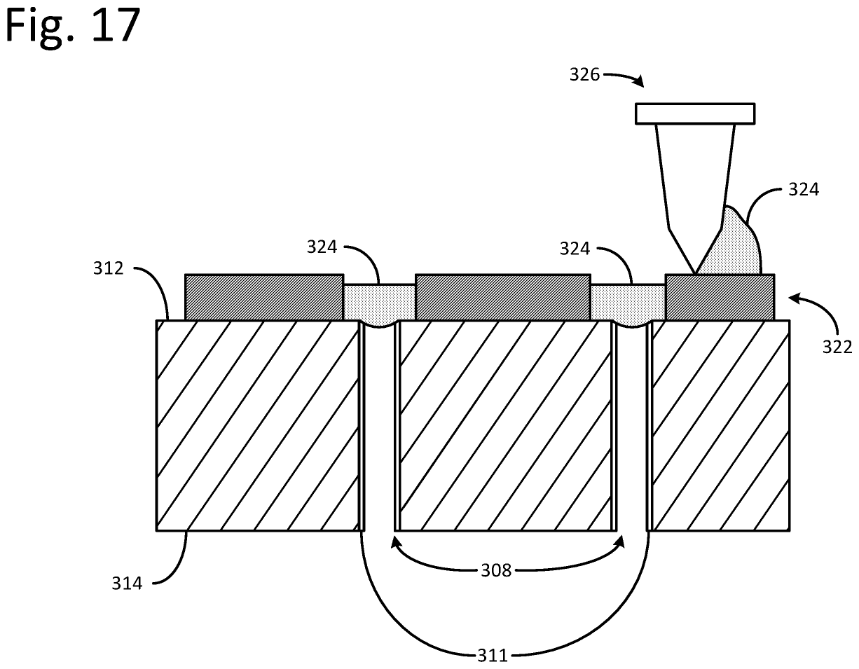

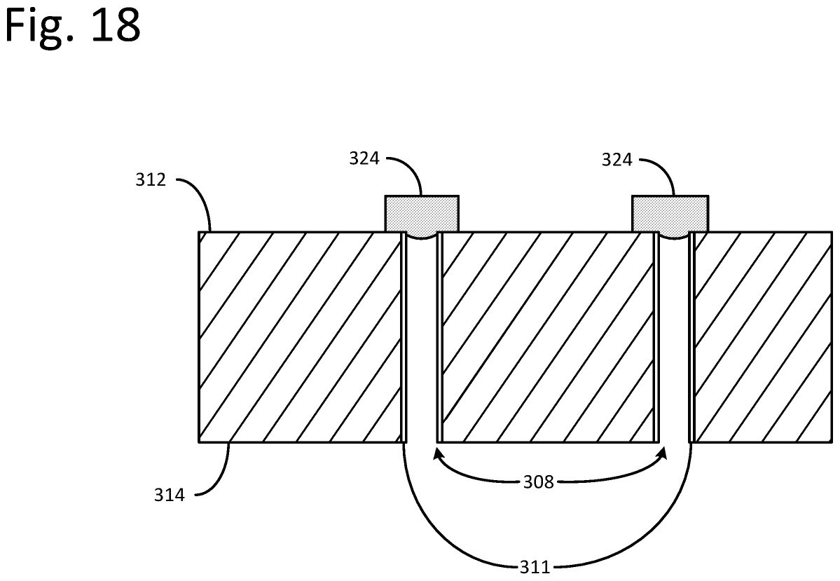

FIGS. 16-18 show a stencil-printing process being used to dispose the gold alloy 310 on the substrate 306. The stencil-printing process may include positioning a stencil 322 on the outer surface 312 of the substrate 306, disposing alloy paste 324 at one side of the stencil 322, pulling the alloy paste 324 across the stencil 322 and the substrate 306, and removing the stencil 322 from the substrate 306. FIG. 16 shows the stencil-printing process after the alloy paste 324 has been disposed but before the paste has been pulled across the stencil 322 and the substrate 306. FIG. 17 shows the stencil-printing process after the alloy paste 324 has been pulled across the stencil 322 and the substrate 306 but before the stencil has been removed from the substrate 306. FIG. 18 shows the substrate 306 and the alloy paste 324 after the stencil has been removed and the stencil-printing process is complete.

The stencil 322 may be used to position the alloy paste 324 relative to the vias 308 prior to a reflowing step or process. For example, the stencil 322 may include openings 323 at or near the position of the vias 308. The openings 323 may allow the alloy paste 324 to be deposited in the vias 308 or on the outer surface 312 of the substrate 306 proximal to the vias 308. The stencil 322 may include any suitable materials such as, for example, stainless steel, nickel, etc. In at least one embodiment, the stencil 319 includes stainless steel.

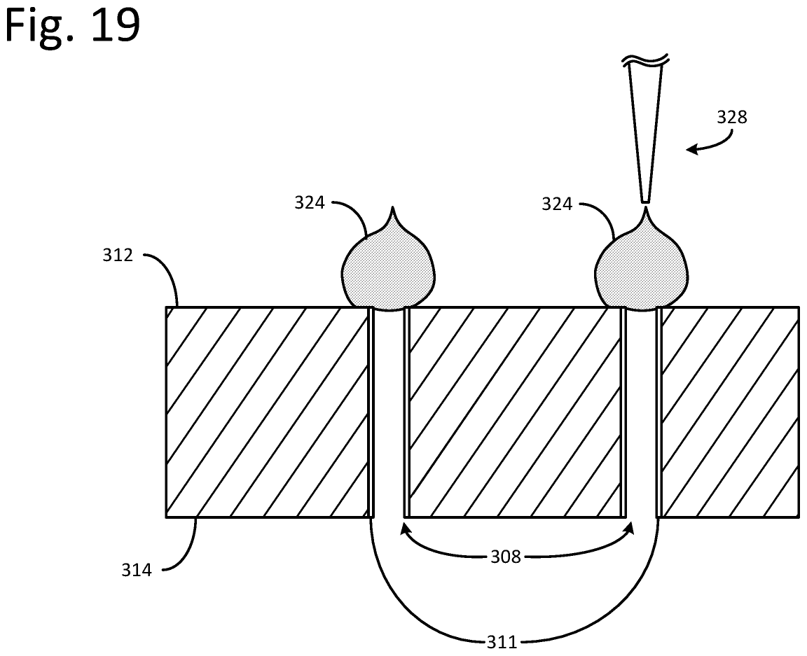

The alloy paste 324 may include the gold alloy 310. The gold alloy 310 may be included in the alloy paste 324 as alloy particles. The alloy paste 324 can include binding agents to hold the alloy particles together. The alloy paste 324 can include any suitable binding agents, e.g., organic binders, solvents, etc. The alloy paste 324 can be dispensed using any suitable dispensing tools and/or nozzles such as, for example, dispenser 328 (see FIG. 19).

The alloy paste 324 can be pulled across the stencil 322 and the substrate 306 using any suitable tool or tools. For example, the alloy paste 324 can be pulled across the stencil 322 and the substrate 306 using a squeegee 326. The squeegee 326 may be configured to pull the alloy paste 324 across the stencil 322 and the substrate 306 causing the alloy paste 324 to be deposited in the openings 323 of the stencil 322. After the alloy paste 324 has been deposited in the openings 323 of the stencil 322, the stencil 322 can be removed from the outer surface 312 of the substrate 306 leaving the alloy paste 324 positioned in and proximal to the vias 308. Accordingly, the substrate 306 and the alloy paste 324 of FIG. 18 may be ready for a reflow process for reflowing the gold alloy 310 into the vias 308.

FIG. 19 shows a dispensing process being used to dispose the gold alloy 310 on the substrate 306. As shown, the dispenser 328 is used to dispense alloy paste 324 onto the substrate 306 proximal to, over, and in the vias 308. The dispenser 328 may be configured to dispense an amount of the alloy paste 324 sufficient to fill the vias 308 with the gold alloy 310 during a reflow process. Accordingly, the substrate 306 and the alloy paste 324 of FIG. 19 may be ready for a reflow process for reflowing the gold alloy 310 into the vias 308.

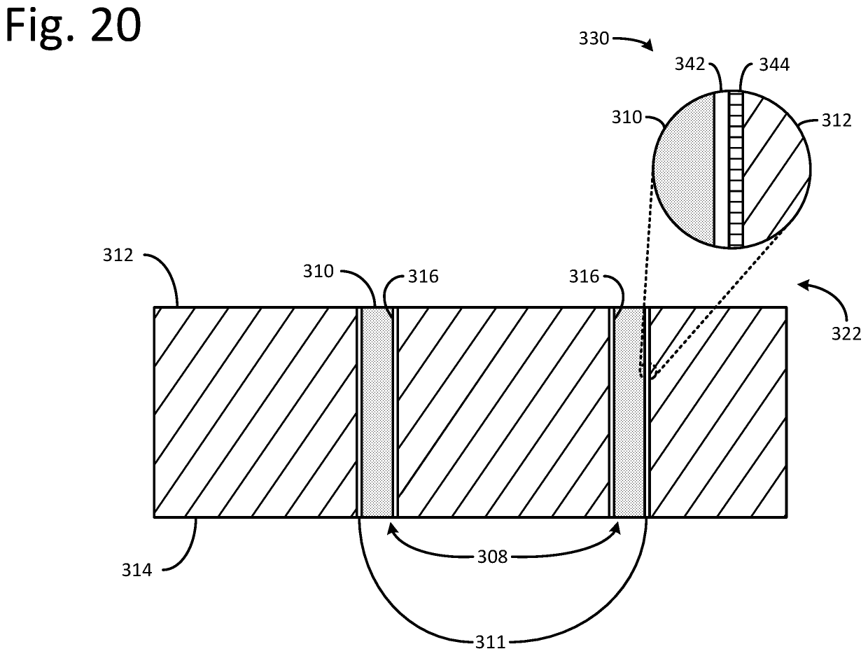

FIG. 20 shows the electrical component 302 after reflowing the gold alloy 310 into the vias 308. The gold alloy 310 filling the vias 308 as depicted in FIG. 20 can be produced by reflowing the alloy bumps 320 of FIG. 14 or the alloy paste 324 of FIGS. 15-18. The interface layer 311 includes the adhesion layer 344 bonded to the substrate 306 and the wettable layer 342 disposed on and bonded the adhesion layer 344 as shown in zoomed portion 330. The bond between the adhesion layer 344 and the substrate 306 may be a covalent bond. In contrast, the bond between the wettable layer 342 and the adhesion layer 344 may be a metallic bond. Such bonds may provide a hermetic seal.

Additionally, the gold alloy 310 may bond with the adhesion layer 342. The wettable layer 342 may be configured to bond to the gold alloy 310 during a reflow process. Accordingly, the gold alloy 310, the interface layer 311, and the sidewalls 316 may be bonded together to form a hermetic seal.

Still further, during the reflow process, the wettable layer 342 may go into solution. In other words, the wettable layer 342 may diffuse and mix with the gold alloy 310 and any materials of the wettable layer 342 may be incorporated into the gold alloy 310 during reflow.

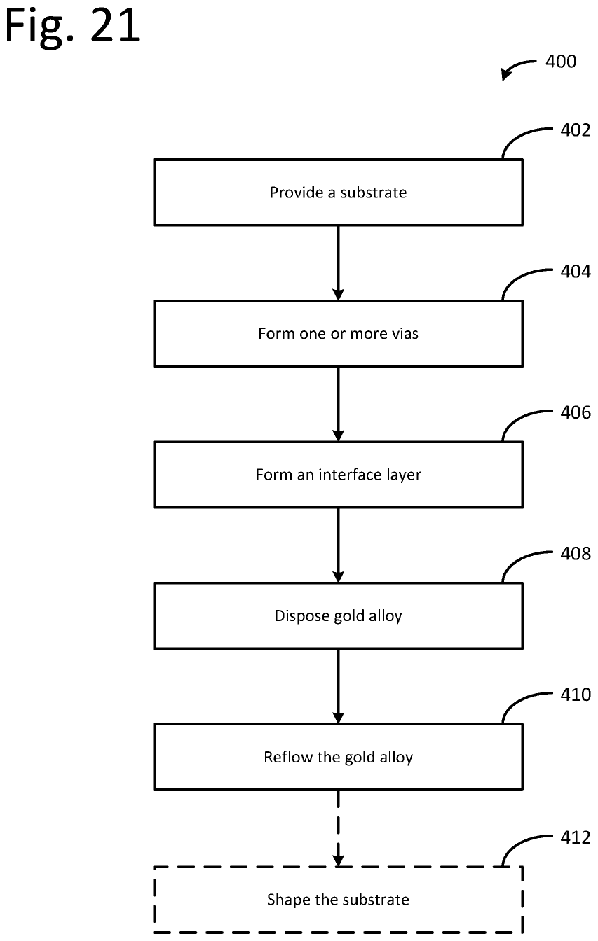

FIG. 21 shows a schematic flow diagram of a method or process 400 for forming an electrical component such as, for example, electrical component 302. Although described in reference to electrical component 302 of FIGS. 11 and 20, the process 400 may be used for any suitable electrical component that includes a substrate, one or more vias, and a gold alloy disposed in the vias.

At 402, the substrate 306 may be provided (see FIG. 12). The substrate may include ceramic, sapphire, glass, or semiconductor materials. Providing the substrate 306 may include shaping, grinding, or polishing to form the outer surface 312 and the outer surface 314.

At 404, one or more vias 308 may be formed in the substrate 306 (see FIG. 13). Each of the one or more vias 308 may include an opening 317 at an outer surface 312 of the substrate 306. Additionally, each of the one or more vias 308 may include a sidewall 316 formed by the substrate 306. The one or more vias 308 may be formed using any suitable technique or techniques. For example, the one or more vias may be formed using laser or mechanical drilling.

At step 406, the interface layer 311 may be formed on at least a portion of the sidewall 316 of each of the one or more vias 308. The interface layer 311 may be formed using any suitable technique or techniques. For example, the interface layer 311 may be formed using sputtering, physical vapor deposition, chemical vapor deposition, atomic layer deposition, or plating (e.g., electroplating or electroless plating). The interface layer 311 may be formed in stages or layers. For example, the adhesion layer 344 may be formed on at least a portion of the sidewall 316 of each of the one or more vias 308. The adhesion layer 344 may be formed using sputtering. Furthermore, the wettable layer 342 may be formed on the adhesion layer 344. The wettable layer 342 may be formed using, for example, physical vapor deposition, chemical vapor deposition, atomic layer deposition, or plating (e.g., electroplating or electroless plating). Each of the wettable layer 342 and the adhesion layer 344 may be formed using any suitable technique or techniques as described above with regard to the interface layer 311. Additionally, the wettable layer 342 may also be formed by plating.

At step 408, the gold alloy 310 may be disposed in the one or more vias 308 or on the outer surface 312 of the substrate 306 proximal to the one or more vias 308. As used herein, the term “proximal to the one or more vias” means that the gold alloy 110 is disposed such that at least a portion of the alloy can flow into one or more openings 117 of the one or more vias 108 when the alloy is melted. Additionally, the gold alloy may be disposed on the outer surface 312 of the substrate such that the opening 317 of each of the one or more vias 308 is at least partially covered by the gold alloy 310.

The gold alloy 310 may be disposed using any suitable technique or techniques. For example, disposing the gold alloy may include, but is not limited to, disposing a bump array 318 of the gold alloy 310 on the outer surface 312 of the substrate 306 such that the opening 317 of each of the one or more vias 308 is at least partially covered by an alloy bump 320 of the bump array 318 (see FIG. 15). The alloy bumps 320 of the bump array 318 may be held in place by a stencil 319 while the bump array 318 is disposed on the outer surface 312 of the substrate 306. Further, for example, disposing the gold alloy 310 may include stencil-printing alloy paste 324 including the gold alloy 310 in the one or more vias 308 or on the outer surface 312 of the substrate 306 proximal to the one or more vias 308 (see FIGS. 16-18). Still further, for example, disposing the gold alloy comprises dispensing an alloy paste comprising the gold alloy in the one or more vias or on the outer surface of the substrate proximal to the one or more vias (see FIG. 19). Additionally, other techniques for disposing via fill material may be used to dispose the gold alloy 310 in the one or more vias 308 or on the outer surface 312 of the substrate 306 proximal to the one or more vias 308.

At 410, the gold alloy 310 may be reflowed into the one or more vias 308. Reflowing the gold alloy 310 may include reducing an atmospheric pressure around the substrate 306 and the gold alloy 310. The atmospheric pressure may be reduced to any suitable level, for example, to less than 10⁻³ Torr. In one or more embodiments, the atmospheric pressure may not be reduced. In one or more embodiments, the atmosphere may be selected to reduce the oxidation state of the alloy constituents or wettable surface, e.g., by utilizing a hydrogen atmosphere.

Reflowing the gold alloy 310 may include brazing the substrate 306 and the gold alloy 310. Reflowing the gold alloy 310 may include heating the substrate 306 and the gold alloy 310 to a peak temperature. Peak temperatures for reflowing the gold alloy 310 may be based on a melting temperature (Tm) of the gold alloy 310. The melting temperature (Tₘ) may also be referred to as the eutectic temperature (T_{E}). Reflowing the gold alloy 310 may include heating the substrate 306 and the gold alloy 310 to a peak temperature, for example, of at least 10 degrees Celsius greater than the melting temperature of the gold alloy 310 and no greater than 100 degrees Celsius greater than the melting temperature of the gold alloy 310 or to a peak temperature within any suitable range therebetween. For example, reflowing the gold alloy 310 may include heating the substrate 306 and the gold alloy 310 to a peak temperature of at least 10 degrees Celsius, 15 degrees Celsius, 20 degrees Celsius, 25 degrees Celsius, 30 degrees Celsius, 35 degrees Celsius, or 40 degrees Celsius greater than the melting temperature of the gold alloy 310 to no greater than 70 degrees Celsius, 75 degrees Celsius, 80 degrees Celsius, 85 degrees Celsius, 90 degrees Celsius, 95 degrees Celsius, or 100 degrees Celsius greater than the melting temperature of the gold alloy 310. In one embodiment, reflowing the gold alloy 310 may include heating the substrate 306 and the gold alloy 310 to a peak temperature of at least 30 degrees Celsius greater than the melting temperature of the gold alloy 310 and no greater than 300 degrees Celsius greater than the melting temperature of the gold alloy 310.

reflowing the gold alloy 310 may include heating the substrate 306 and the gold alloy 310 to a peak temperature independent of the melting temperature. In at least one embodiment, reflowing the gold alloy 310 includes heating the gold alloy 310 to a peak temperature of at least 330 degrees Celsius and no greater than 390 degrees Celsius. The substrate 306 and the gold alloy 310 may be heated at the peak temperature for at least 10 seconds and no greater than 30 minutes or for any suitable range of time therebetween. For example, the substrate 306 and the gold alloy 310 may be heated at the peak temperature in a range from at least 10 seconds, 20 seconds, 30 seconds, 40 seconds, 50 seconds, 1 minute to no greater than 3 minutes, 5 minutes, 10 minutes, 15 minutes, 20 minutes, 25 minutes, or 30 minutes. In at least one embodiment, the gold alloy 310 may be heated at the peak temperature for at least 1 minute and no greater than 15 minutes.

Reflowing the gold alloy 310 may include filling a volume surrounding the substrate 306 and the gold alloy 310 with an inert gas. The volume may include an inner volume of a furnace for heating the substrate 306 and the gold alloy 310. The inert gas may include, for example, argon, nitrogen, Diazene (N₂H₂), or mixtures thereof. In at least one embodiment, the inert gas includes argon.

At 412, the substrate 306 may be shaped. Shaping the substrate 306 may include grinding or polishing one or more surfaces of the substrate 306. Additionally, shaping of the substrate 306 may include grinding or polishing the one or more vias 308. Shaping the substrate 306 may smooth portions of the outer surfaces 312, 314 of the substrate 306 and/or the one or more vias 308. Shaping the substrate 306 may result in one or more planar or curved surfaces.

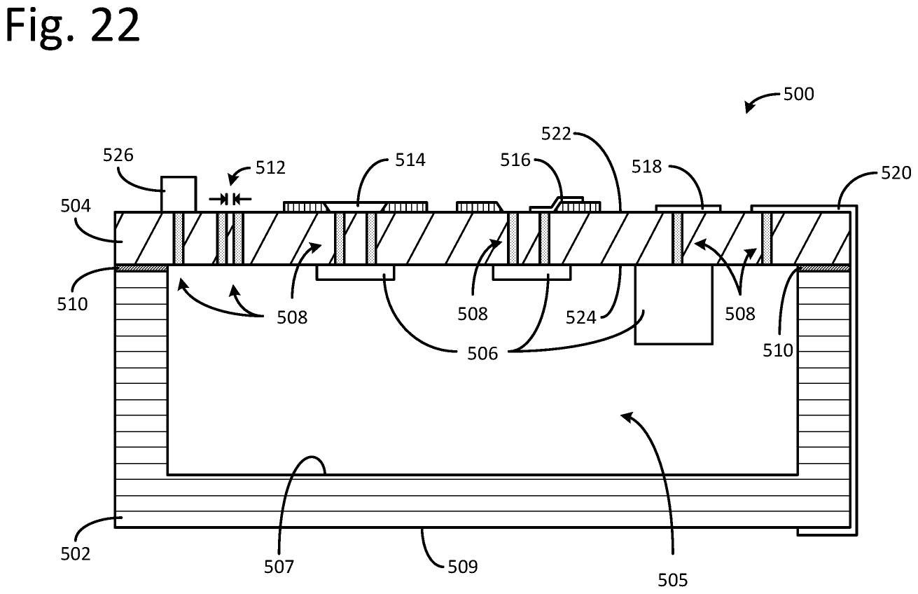

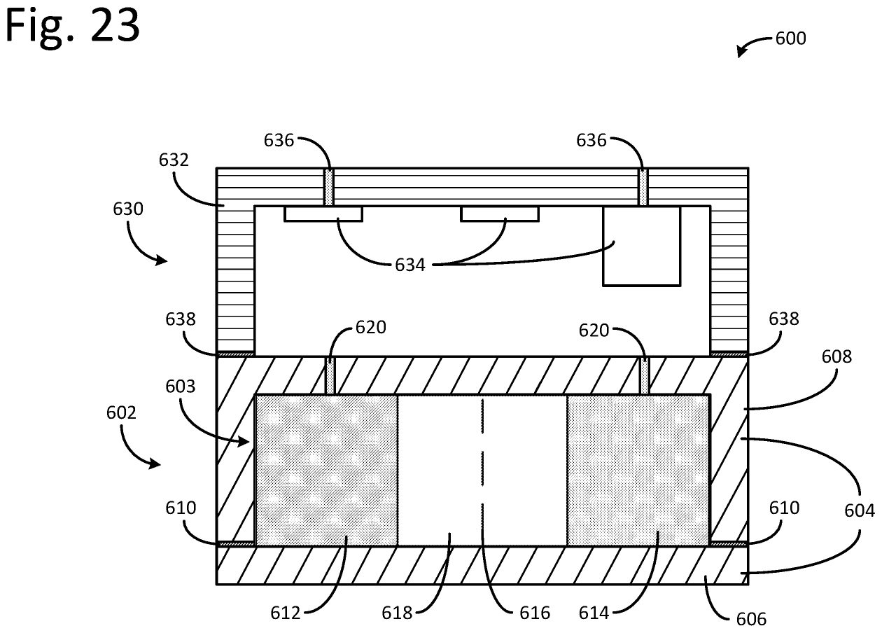

The preceding methods and processes described herein may be used in hermetically-sealed packages and systems such as those depicted in FIGS. 22 and 23. FIG. 22 shows a schematic cross-sectional view of a hermetically-sealed package 500 that includes corrosion-resistant vias as described herein. The hermetically-sealed package 500 may be a remote sensing device, a chemical sensing device, a chemical processing device, an implantable medical device, or other device that may be subject to corrosive environments.

The hermetically-sealed package 500 includes a housing 502, one or more electronic devices 506 disposed in the housing 502, a substrate 504, and corrosion-resistant vias 508 disposed in the substrate 504. The housing 502 may include an inner surface 507 and an outer surface 509.

The housing 502 may include any suitable material or materials, e.g., metallic, polymeric, ceramic, or inorganic materials. In one or more embodiments, the housing 502 can include at least one of glass, quartz, silica, sapphire, silicon carbide, diamond, MP35N (available from Elgiloy Specialty Metals, Sycamore, IL), or gallium nitride. In one or more embodiments, the housing 502 may include at least one of copper, silver, titanium, niobium, zirconium, tantalum, stainless steel, platinum, or iridium. In one or more embodiments, the housing 502 may include the same material or materials as the substrate 504. Further, in one or more embodiments, the housing 502 may include biocompatible materials such that the package 500 can be implanted within a patient's body. Further, one or more coatings or layers can be disposed on the outer surface 509 of the housing that provide biocompatibility. In one or more embodiments, the housing 502 can be electrically conductive to provide a ground electrode for the package 500. In one or more embodiments, the housing 502 can be nonconductive.

The housing 502 can take any suitable shape or shapes and have any suitable dimensions. In one or more embodiments, the housing 502 takes a shape that forms the cavity 505 that can accommodate the electronic devices 506 disposed in the housing. The cavity 505 may be defined by the inner surface 507 of the housing 502 and one or more surfaces of the substrate 504. As shown, the cavity 505 is defined by the inner surface 507 of the housing 502 and a second major surface 524 of the substrate 504. The housing 502 can be a single, unitary housing. In one or more embodiments, the housing 502 can include two or more parts that are made separately and then connected together using any suitable technique or techniques.

Sealed to the housing 502 is the substrate 504. The substrate 504 may include a thickness extending between a first major surface 522 and the second major surface 524. The substrate 504 can take on any suitable properties or characteristics described herein with regard to the substrates 106 and 306 (see FIGS. 1-9 and 11-20).

The substrate 504 can be sealed to the housing 502 using any suitable technique or techniques, e.g., mechanically fastening, adhering, press fitting, laser bonding, magnetic coupling, etc. In one or more embodiments, the substrate 504 is sealed to the housing 502 using a bonding layer 510. The bonding layer 510 can include any suitable corrosion-resistant material or materials such as, for example, titanium, niobium, zirconium, tantalum, stainless steel, platinum, iridium, or combinations thereof. In one or more embodiments, the bonding layer 510 may include a dielectric material or materials to provide a dielectric bonding ring, e.g., the same materials described herein regarding the substrate 504. Further, the bonding layer 510 may take any suitable shape and have any suitable dimensions. The bonding layer 510 may be disposed between the first major surface 522 of the substrate 504 and the housing 502. In one or more embodiments, the bonding layer 510 can be hermetically sealed to one or both of the first major surface 522 of the substrate 504 and the housing 502. The bonding layer 510 can be sealed to one or both of the substrate 504 and the housing 502 using any suitable technique or combination of techniques, e.g., laser bonding. In one or more embodiments, the bonding layer 510 is first attached to the first major surface 522 of the substrate 504 and then attached to the housing 502. In one or more embodiments, the bonding layer 510 is first attached to the housing 502 and then to the second major surface 524 of the substrate 504.

In one or more embodiments, the bonding layer 510 can be hermetically sealed to the housing 502 using any suitable technique or techniques, e.g., the techniques described in co-owned and co-filed U.S. patent application Ser. No. 14/976,475 to Sandlin et al. and entitled KINETICALLY LIMITED NANO-SCALE DIFFUSION BOND STRUCTURES AND METHODS. In one or more embodiments, electromagnetic radiation (e.g., light) can be directed through the bonding layer 510 from its first major surface 522 and focused at a region between the second major surface 524 and the housing 502. Any suitable electromagnetic radiation can be utilized to form the bond. In one or more embodiments, the electromagnetic radiation can include laser light that can include any suitable wavelength or range of wavelengths. In one or more embodiments, the laser light can include light having a wavelength of at least 200 nm. In one or more embodiments, the laser light can include a wavelength of no greater than 2000 nm. For example, laser light can include UV light, visible light, IR light, and combinations thereof. The UV light can be provided by a UV laser that has any suitable wavelength or range of wavelengths and any suitable pulse width. In one or more embodiments, a UV laser can be utilized to provide light having a wavelength in a range of 100-400 nm and a pulse width in a range of 1-100 ns. In one or more embodiments, the materials for the substrate 504 and the housing 502, and the power level and wavelength of the light used may be selected such that the light may not directly damage, ablate, warp, or cut the substrate and the housing, and such that the substrate and the housing retain their bulk properties.

In general, light can be provided by any suitable laser or laser system. For example, the laser may generate light having a relatively narrow set of wavelengths (e.g., a single wavelength). In one or more embodiments, the light emitted by the laser may form a collimated beam that may not be focused at a particular point. In one or more embodiments, the light emitted by the laser may be focused at a focal point at a region between the first major surface 522 of the bonding layer 510 and the housing 502 to generate a laser bond.

Although the laser may provide light that has a narrow range of wavelengths, in one or more embodiments, the laser may represent one or more devices that emit electromagnetic radiation having a wider range of wavelengths than a single typical laser. A wide variety of devices may be used to emit electromagnetic radiation having a narrow or wide range of wavelengths. In one or more embodiments, the laser may include one or more laser devices including diode and fiber lasers. Laser sources may also include, e.g., TI sapphire lasers, argon ion lasers, Nd:YAG lasers, XeF lasers, HeNe lasers, Dye lasers, GaAs/AlGaAs lasers, Alexandrite lasers, InGaAs lasers, InGaAsP lasers, Nd:glass lasers, Yb:YAG lasers, and Yb fiber lasers. The laser device may also include one of continuous wave, modulated, or pulsed modes. Accordingly, a wide variety of laser devices may be used in the bonding process. In one or more embodiments, a power level of the laser may be set to approximately 1 W, distributed across the approximate focused beam diameter of 10 μm, with a top hat, Gaussian, or other suitable spatial energy profile.

The second major surface 524 of the substrate 504 can be bonded to the bonding layer 510 using any suitable technique or techniques. In one or more embodiments, the second major surface 524 of the substrate 504 can be bonded to the bonding layer 510 utilizing typical wafer-to-wafer bonding techniques, e.g., direct bonding, surface activated bonding, anodic bonding, eutectic bonding, glass frit bonding, adhesive bonding, thermocompression bonding, reactive bonding, and transient liquid phase diffusion bonding.

Electrical connections may be provided between the electronic devices 506 disposed in the housing 502 and devices external to the housing by the corrosion-resistant vias 508. The corrosion-resistant vias 508 may be disposed in the substrate 504 using any suitable technique or techniques as described herein with regard to corrosion-resistant vias 108 and 308 (see FIGS. 1-9 and 11-20). The corrosion-resistant vias 508 may include the same materials described herein with regard to corrosion-resistant vias 108 and 308 (see FIGS. 1-9 and 11-20) including. The corrosion-resistant vias 508 may include a corrosion-resistant alloy. The corrosion-resistant alloy of vias 508 may include any of the materials described herein with regard to the corrosion-resistant alloy 110 or gold alloy 310 (see FIGS. 1 and 11).

The corrosion-resistant vias 508 may be corrosion-resistant and biostable without additional processes or components such as, for example, a foil cap. Existing packages and devices may use a foil cap (e.g., titanium foil) to cover vias to prevent corrosion while still providing an electrical connection. Such existing foil caps may have a thickness of about 50 to 250 micrometers. Accordingly, such existing packages do not allow components to be directly coupled to vias with the foil cap and a thickness of the packages is increased. Furthermore, a minimum pitch or distance between the vias of such existing packages is limited by the width of the foil caps. Without a need for foil caps, a minimum pitch 512 of the corrosion-resistant vias 508 may be dependent on the strength of the materials of the substrate 504 and the accuracy or tolerance of manufacturing equipment. The minimum pitch 512 of the corrosion-resistant vias 508 may be less than 200 micrometers.

Additionally, components may be directly coupled to the corrosion-resistant vias 508 and the substrate 504. Components that may be directly attached to the corrosion-resistant vias 508 may include, for example, a well via 514; thin film components 516, 518; an electroplated conductive component 520; and a fixation component 526. The well via 514 may include a well or recess in non-conductive material formed on the first major surface 522 of the substrate 504. The well or recess of the well via 514 may be filled with a corrosion-resistant alloy. The corrosion resistant-alloy of the well via 514 may include the same material or materials used in the corrosion-resistant alloy of the corrosion-resistant vias 508. The well via 514 may include material that responds to an analyte. For example, the well via 514 may be used as an electrode for chemical sensors and may provide an electrical signal in response to contact with the analyte. Without the need for a foil cap, non-planar components and surfaces (e.g., wells, fixation components, etc.) can be disposed directly on the substrate 504 and have a thinner profile or smaller thickness than a foil cap.

The thin film components 516, 518 may also be disposed on the substrate 504. Such thin film components 516, 518 may be directly coupled to the corrosion-resistant vias 508 and have a thinner profile or smaller thickness than a foil cap. Each of the thin film components 516, 518 may include an electrode, an antenna, a conductive trace, etc. The thin film component 516 may extend out of a well formed by non-conductive material. The thin film component 518 may form a layer on the first major surface 522 of the substrate 504. The thin film components 516, 518 may include corrosion-resistant materials such as gold, titanium, niobium, zirconium, tantalum, stainless steel, platinum, iridium, or combinations thereof. The thin film components 516, 518 may have a thickness of at least 100 nanometers and no greater than 10 micrometers. In at least one embodiment, the thin film components 516, 518 have a thickness of 0.5 micrometers to about 3 micrometers.

The thin film components 516, 518 can be electrically connected to the vias 508 using any suitable technique. In one or more embodiments, the thin film components 516, 518 can be bonded to the substrate 504 using any suitable technique (e.g., laser bonding) such that the components are electrically connected to the vias 508. In one or more embodiments, the thin film components 516, 518 can be hermetically sealed to the substrate 504 using any suitable technique (e.g., laser bonding) to assist in preventing ingress of fluid into the vias 508.

In addition to thin film components 516, 518, electrodes, antennas, and conductive traces may be electroplated such as electroplated conductive component 520. The electroplated conductive component 520 may include corrosion-resistant materials such as titanium, niobium, zirconium, tantalum, rhodium, platinum, iridium, or combinations thereof. The electroplated conductive component 520 may have a thickness of at least 100 nanometers and no greater than 10 micrometers. In at least one embodiment, the electroplated conductive component 520 has a thickness of 0.5 micrometers to about 3 micrometers. Furthermore, the electroplated conductive component 520 can be plated to multiple sides of the substrate 504 and the housing 502.

Fixation elements such as fixation component 526 may be directly coupled to the corrosion-resistant vias 508. The fixation component 526 may be coupled to the corrosion-resistant vias 508 during a reflow step or process such as the reflow steps 208 and 410 of methods 200 and 400 (see FIGS. 10 and 21). The fixation component 526 may include, for example, metal block, brackets, or other component for welding wires to.

Additional components that may be directly attached to the corrosion-resistant vias 508 may include, for example, capacitors, transistors, integrated circuits, including controllers and multiplexers, sensors, batteries, etc. Such components may be disposed on any surfaces of the substrate 504 or the housing 502. Furthermore, any suitable technique or techniques can be utilized to dispose the components on the substrate 504 or the housing 502, for example, soldering, brazing, plating, etc.

In addition to the corrosion-resistant vias 508 allowing components to be directly coupled to them while keeping the hermetically-sealed package 500 corrosion-resistant and hermetically-sealed, such corrosion-resistant vias may also provide an effective seal for battery chemistries as depicted in FIG. 23. FIG. 23 shows a schematic cross-sectional view of a system 600 that includes a hermetically-sealed battery 602.

The hermetically-sealed battery 602 may include a battery housing 604, an electrochemical cell 603 disposed in the battery housing, and electrodes 620. The battery housing 604 may include any suitable materials such as those described herein with regard to the substrates 106 and 306 of the electrical components 102 and 302. The battery housing 604 may include a first portion 606 and a second portion 608 hermetically sealed to each other by a bonding layer 610. The bonding layer 610 may include any suitable materials and take on any suitable shape as described herein with regard to bonding layer 510 (see FIG. 21). Furthermore, the bonding layer 610 may be bonded or sealed to the first portion 606 and the second portion 608 of the battery housing using any suitable technique or techniques as described herein with regard to bonding layer 510. Although shown as two portions, the battery housing 604 can include any number of portions sealed together by one or more bonding layers such as bonding layer 510. The battery housing 604 can take any suitable shape or shapes and have any suitable dimensions. In one or more embodiments, the battery housing 604 takes a shape that forms a cavity that can accommodate the electrochemical cell 603 disposed in the housing.

The electrochemical cell 603 includes an anode 612, a cathode 614, an electrolyte 618, and a separator 616 (e.g., a polymeric microporous separator, indicated by the dashed line) provided intermediate or between the anode 612 and the cathode 614. The anode 612 and the cathode 614 may be provided as relatively flat or planar plates or may be wrapped or wound in a spiral or other configuration (e.g., an oval configuration). The anode 612 and the cathode 614 may also be provided in a folded configuration.

The electrochemical cell 603 may include any suitable chemistry. The chemistry of the electrochemical cell 603 may include, for example, lithium-metal, lithium-ion, lithium polymer, zinc/silver oxide, or other battery chemistry. In at least one embodiment, the electrochemical cell 603 includes a lithium-metal battery cell. The electrochemical cell 603 may be a primary cell or a secondary cell. In other words, the electrochemical cell 603 may or may not be rechargeable.

The electrolyte 618 may transport positively charged ions between the cathode 614 and the anode 612. During charging and discharging of the electrochemical cell 603, positively charged ions may move between the anode 612 and the cathode 614. For example, when the electrochemical cell 603 is discharged, positively charged ions flow from the cathode 614 to the anode 612. In contrast, when the electrochemical cell 603 is charged, lithium ions flow from the anode 612 to the cathode 614. The separator 616 may be arranged to prevent direct contact between the anode and the cathode. However, the separator 616 allows the flow of positively charged ions between the cathode 614 and the anode 612.

To allow the electrochemical cell 603 to provide power to electronic devices, the hermetically-sealed battery 602 may include electrodes 620. Each of the electrodes 620 may include a corrosion-resistant via disposed in the battery housing 604. A first electrode of the electrodes 620 may be electrically coupled to the anode 612 of the electrochemical cell 603. A second electrode of the electrodes 620 may be electrically coupled to the cathode 614 of the electrochemical cell. Each of the corrosion-resistant vias that form the electrodes 620 may include one or more sidewalls formed by the battery housing 604, a corrosion-resistant alloy, and a hermetic and corrosion-resistant seal formed between the corrosion-resistant alloy and the one or more sidewalls. The corrosion-resistant vias of the electrodes 620 may be disposed in the battery housing 604 using any suitable technique or techniques as described herein with regard to corrosion-resistant vias 108 and 308 (see FIGS. 1-9 and 11-20). Furthermore, the corrosion-resistant vias of the electrodes 620 may include the same materials described herein with regard to corrosion-resistant vias 108 and 308 (see FIGS. 1-9 and 11-20). Still further, the corrosion-resistant alloy of the corrosion-resistant vias may include any of the materials described herein with regard to the corrosion-resistant alloy 110 or gold alloy 310 (see FIGS. 1 and 11).

In addition to the hermetically-sealed battery 602, the system 600 may further include a package 630 hermetically sealed to the hermetically-sealed battery 602. The package 630 may include a package housing 632, one or more electronic devices 634 disposed inside the package housing, and one or more corrosion-resistant vias 636 disposed in the package housing. In general, the package 630 may include the devices and apparatus as described herein with regard to package 500 of FIG. 22.

The package housing 632 may include transparent ceramic, sapphire, or glass. Furthermore, the package housing 632 may be hermetically sealed to the battery housing. The package housing 632 may be hermetically sealed to the battery housing using a bonding layer 638. The bonding layer 638 may include any suitable materials and take on any suitable shape as described herein with regard to bonding layer 510 of FIG. 22. Furthermore, the bonding layer 638 may be bonded or sealed to the battery housing 604 and the package housing 632 using any suitable technique or techniques as described herein with regard to bonding layer 510.

The electronic devices 634 may be electrically coupled to the electrochemical cell 603 via electrodes 620. In other words, the hermetically-sealed battery 602 may provide power to the electronic devices 634 via the electrodes 620. The electronic devices 634 may be electrically coupled to the electrodes 620 using any suitable techniques or apparatus. For example, one or more conductive traces, springs, friction fit elements, curable traces, or other conductive apparatus may electrically couple the electronic devices 634 to the electrodes 620. Conductive springs may allow for a blind electrical connection between the electronic devices 634 and the electrodes 620 when the package housing 632 is attached to the battery housing 604. Curable conductive traces may also allow for an electrical connection to be formed between the electronic devices 634 and the electrodes 620 after the package housing 632 is attached to the battery housing 604. Such curable conductive traces may be cured after the package housing 632 is attached to the battery housing 604 by a laser, heat, or other curing process.

Furthermore, the electronic devices 634 may be electrically coupled to external components or devices by corrosion-resistant vias 636. The corrosion-resistant vias 636 may include one or more sidewalls formed by the package housing 632, a corrosion-resistant alloy, and a hermetic and corrosion-resistant seal formed between the corrosion-resistant alloy and the one or more sidewalls. The corrosion-resistant vias 636 may be disposed in the package housing 632 using any suitable technique or techniques as described herein with regard to corrosion-resistant vias 108 and 308 (see FIGS. 1-9 and 11-20). Furthermore, the corrosion-resistant vias 636 may include the same materials described herein with regard to corrosion-resistant vias 108 and 308 (see FIGS. 1-9 and 11-20). Still further, the corrosion-resistant alloy of the corrosion-resistant vias may include any of the materials described herein with regard to the corrosion-resistant alloy 110 or gold alloy 310 (see FIGS. 1 and 11).

The invention is defined in the claims. However, below there is provided a non-exhaustive listing of non-limiting examples. Any one or more of the features of these examples may be combined with any one or more features of another example, embodiment, or aspect described herein.

Example Ex1. A hermetically-sealed package that includes a housing, one or more electronic devices disposed inside the housing, a substrate, and one or more corrosion-resistant vias disposed in the substrate. The substrate includes ceramic, sapphire, or glass. The substrate is hermetically sealed to the housing. Each of the one or more corrosion-resistant vias includes one or more sidewalls formed by the substrate, a corrosion-resistant alloy, and a hermetic and corrosion-resistant seal formed between the corrosion-resistant alloy and the one or more sidewalls.

Example Ex2. The hermetically-sealed package of Ex1, where the hermetic and corrosion-resistant seal formed between the corrosion-resistant alloy and the one or more sidewalls includes a reaction layer bonding the corrosion-resistant alloy and the one or more sidewalls.

Example Ex3. The hermetically-sealed package of Ex2, where the corrosion-resistant alloy includes zirconium.

Example Ex4. The hermetically-sealed package of Ex3, where the corrosion-resistant alloy includes 58 to 64.9 percent by weight zirconium.

Example Ex5. The hermetically-sealed package of Ex1, where the hermetic and corrosion-resistant seal formed between the corrosion-resistant alloy and the one or more sidewalls includes an interface layer bonding the corrosion-resistant alloy to the substrate.

Example Ex6. The hermetically-sealed package of Ex5, where the corrosion-resistant alloy includes a gold alloy.

Example Ex7. The hermetically-sealed package of Ex1, further including a thin film component disposed on a surface of the substrate.

Example Ex8. The hermetically-sealed package of Ex7, where the thin film component includes an antenna.

Example Ex9. The hermetically-sealed package of Ex7, where the thin film component includes an electrode.

Example Ex10. The hermetically-sealed package of Ex7, where the thin film component has a thickness of at least 0.5 micrometers and no greater than 3 micrometers.

Example Ex11. The hermetically-sealed package of Ex1, further including a fixation element directly coupled to one of the one or more corrosion-resistant vias.

Example Ex12. The hermetically-sealed package of Ex1, further including a continuous electroplated conductive element disposed on an outer surface of the housing and an outer surface of the substrate.

Example Ex13. The hermetically-sealed package of Ex1, where the hermetically-sealed package includes an implantable medical device.

Example Ex14. A system including a hermetically-sealed battery. The hermetically-sealed battery includes a battery housing, an electrochemical cell, a first electrode, and a second electrode. The battery housing includes ceramic, sapphire, or glass. The electrochemical cell is disposed within the battery housing. The electrochemical cell includes an anode, a cathode, a separator arranged between the anode and the cathode, and an electrolyte. The separator is configured to prevent direct contact between the anode and the cathode. The electrolyte transports positively charged ions between the cathode and the anode. The first electrode includes a corrosion-resistant via disposed in the battery housing and electrically coupled to the anode of the electrochemical cell. The second electrode includes a corrosion-resistant via disposed in the battery housing and electrically coupled to the cathode of the electrochemical cell. The corrosion-resistant via of the first electrode and the corrosion-resistant via of the second electrode each includes one or more sidewalls formed by the battery housing, a corrosion-resistant alloy, and a hermetic and corrosion-resistant seal formed between the corrosion-resistant alloy and the one or more sidewalls formed by the battery housing.

Example Ex15. The system of Ex14, further including a package hermetically sealed to the hermetically-sealed battery.

Example Ex16. The system of Ex15, where the package includes a package housing including ceramic, sapphire, or glass, the package housing hermetically sealed to the battery housing; one or more electronic devices disposed inside the package housing; and one or more corrosion-resistant vias disposed in the package housing. Each of the one or more corrosion-resistant vias includes one or more sidewalls formed by the package housing; a corrosion-resistant alloy; and a hermetic and corrosion-resistant seal formed between the corrosion-resistant alloy and the one or more sidewalls formed by the package housing.

Example Ex17. The system of Ex14, where the hermetic and corrosion-resistant seal formed between the corrosion-resistant alloy and the one or more sidewalls includes a reaction layer bonding the corrosion-resistant alloy and one or more sidewalls formed by the battery housing.

Example Ex18. The system of Ex14, where the corrosion-resistant alloy includes zirconium.

Example Ex19. The system of Ex14, where the corrosion-resistant alloy includes 58 to 64.9 percent by weight zirconium.

Example Ex20. The system of Ex14, where the hermetic and corrosion-resistant seal formed between the corrosion-resistant alloy and the one or more sidewalls includes an interface layer bonding the corrosion-resistant alloy to the one or more sidewalls formed by the battery housing.

All references and publications cited herein are expressly incorporated herein by reference in their entirety into this disclosure, except to the extent they may directly contradict this disclosure. Illustrative embodiments of this disclosure are discussed, and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

What is claimed is:

1. A hermetically-sealed package comprising:
a housing;
one or more electronic devices disposed in the housing;
a substrate comprising ceramic, sapphire, or glass, the substrate hermetically sealed to the housing; and
one or more corrosion-resistant vias disposed in the substrate, each of the one or more corrosion-resistant vias comprising:
one or more sidewalls formed by the substrate;
a corrosion-resistant alloy; and
a hermetic and corrosion-resistant seal formed between the corrosion-resistant alloy and the one or more sidewalls.

2. The hermetically-sealed package of claim 1, wherein the hermetic and corrosion-resistant seal formed between the corrosion-resistant alloy and the one or more sidewalls comprises a reaction layer bonding the corrosion-resistant alloy and the one or more sidewalls.

3. The hermetically-sealed package of claim 2, wherein the corrosion-resistant alloy comprises zirconium.

4. The hermetically-sealed package of claim 3, wherein the corrosion-resistant alloy comprises 58 to 64.9 percent by weight zirconium.

5. The hermetically-sealed package of claim 1, wherein the hermetic and corrosion-resistant seal formed between the corrosion-resistant alloy and the one or more sidewalls comprises an interface layer bonding the corrosion-resistant alloy to the substrate.

6. The hermetically-sealed package of claim 5, wherein the corrosion-resistant alloy comprises a gold alloy.

7. The hermetically-sealed package of claim 1, further comprising a thin film component disposed on a surface of the substrate.

8. The hermetically-sealed package of claim 7, wherein the thin film component comprises an antenna.

9. The hermetically-sealed package of claim 7, wherein the thin film component comprises an electrode.

10. The hermetically-sealed package of claim 7, wherein the thin film component has a thickness of at least 0.5 micrometers and no greater than 3 micrometers.

11. The hermetically-sealed package of claim 1, further comprising a fixation element directly coupled to one of the one or more corrosion-resistant vias.

12. The hermetically-sealed package of claim 1, further comprising a continuous electroplated conductive element disposed on an outer surface of the housing and an outer surface of the substrate.

13. The hermetically-sealed package of claim 1, wherein the hermetically-sealed package comprises an implantable medical device.

14. A system comprising a hermetically-sealed battery, the hermetically-sealed battery comprising:
a battery housing comprising ceramic, sapphire, or glass;
an electrochemical cell disposed within the battery housing and comprising:
an anode;
a cathode;
a separator arranged between the anode and the cathode, the separator configured to prevent direct contact between the anode and the cathode; and
an electrolyte to transport positively charged ions between the cathode and the anode;
a first electrode comprising a corrosion-resistant via disposed in the battery housing and electrically coupled to the anode of the electrochemical cell; and
a second electrode comprising a corrosion-resistant via disposed in the battery housing and electrically coupled to the cathode of the electrochemical cell;
wherein the corrosion-resistant via of the first electrode and the corrosion resistant via of the second electrode each comprise:
one or more sidewalls formed by the battery housing;
a corrosion-resistant alloy; and
a hermetic and corrosion-resistant seal formed between the corrosion-resistant alloy and the one or more sidewalls formed by the battery housing.

15. The system of claim 14, further comprising a package hermetically sealed to the hermetically-sealed battery.

16. The system of claim 15, wherein the package comprises:
a package housing comprising ceramic, sapphire, or glass, the package housing hermetically sealed to the battery housing;
one or more electronic devices disposed inside the package housing; and
one or more corrosion-resistant vias disposed in the package housing, each of the one or more corrosion-resistant vias comprising:
one or more sidewalls formed by the package housing;
a corrosion-resistant alloy; and
a hermetic and corrosion-resistant seal formed between the corrosion-resistant alloy and the one or more sidewalls formed by the package housing.

17. The system of claim 14, wherein the hermetic and corrosion-resistant seal formed between the corrosion-resistant alloy and the one or more sidewalls comprises a reaction layer bonding the corrosion-resistant alloy and one or more sidewalls formed by the battery housing.

18. The system of claim 14, wherein the corrosion-resistant alloy comprises zirconium.

19. The system of claim 14, wherein the corrosion-resistant alloy comprises 58 to 64.9 percent by weight zirconium.

20. The system of claim 14, wherein the hermetic and corrosion-resistant seal formed between the corrosion-resistant alloy and the one or more sidewalls comprises an interface layer bonding the corrosion-resistant alloy to the one or more sidewalls formed by the battery housing.
